(19) United States
(12) Patent Application Publication
(10) Pub. No.: US 2024/0392350 A1
(43) Pub. Date: Nov. 28, 2024

(54) DNA VALENCY SORTING CHROMATOGRAPHY

(71) Applicant: The Trustees of Princeton University, Princeton, NJ (US)

(72) Inventors: Haw YANG, Princeton Junction, NJ (US); Nyssa EMERSON, Princeton, NJ (US)

(73) Assignee: The Trustees of Princeton University, Princeton, NJ (US)

(21) Appl. No.: 18/694,288

(22) PCT Filed: Sep. 30, 2022
(86) PCT No.: PCT/US2022/045349

§ 371 Date: Mar. 21, 2024

Related U.S. Application Data
(60) Provisional application No. 63/250,333, filed on Sep. 30, 2021.

(51) Int. Cl. C12Q 1/6837; B01L 3/00; C12Q 1/6804

(52) U.S. Cl. CPC C12Q 1/6837; B01L 3/502761; C12Q 1/6804; B01L 2200/0652; B01L 2300/0654

(57) ABSTRACT

Disclosed is DNA valency sorting chromatography, a purification method for separating solutes based on the number of barcoded DNA molecules present on their surface, which can operate using conventional low-pressure chromatography equipment and instrumentation. Solutes can take a variety of forms, including biological macromolecules, polymeric nanoparticles, gold or silver nanospheres, gold nanorods, iron oxide nanoparticles, and semiconducting nanocrystals. In contrast to most existing purification procedures, DNA valency sorting is highly selective for the DNA sequence specifically, rather than the characteristics of the solute as a whole, and uses extremely gentle elution conditions. As a result, it is applicable to a range of solute characteristics, including variable chemical composition, surface charge, and materials with hydrodynamic diameters up to 80 nm, which cannot be purified with a well-defined number of macromolecules by any other existing technique.

### CROSS-REFERENCE TO RELATED APPLICATIONS

The present application claims priority to U.S. Provisional Application 63/250,333, filed Sep. 30, 2021, the entirety of which is incorporated by reference herein.

### REFERENCE TO A SEQUENCE LISTING

The present application contains a Sequence Listing in computer readable form. The computer readable form is incorporated herein by reference.

### TECHNICAL FIELD

The present application is drawn to DNA “barcodes” that can be used for, e.g., chromatography and specifically sorting solutes based on those barcodes.

### BACKGROUND

Nanoparticles bearing a specified number of DNA oligonucleotides are versatile reagents in nanotechnology. The number (valency) and sequence of the attached DNA encode their interactions, allowing them to be programmatically assembled with other DNA-labeled materials to form well-defined products, akin to a chemical reaction. Such DNA-encoded nanochemistry was first demonstrated in 1996 with the synthesis of simple linear dimeric and trimeric gold nanocluster “molecules”. With advancements in DNA nanotechnology, researchers have since assembled DNA valency-defined nanoparticles—primarily small, gold nanospheres—into a rich array of complex, three-dimensional static and dynamic molecule-like structures. The same concept has also been used to interface nanoparticles with a precise number of DNA-labeled proteins or bio-active molecules. These examples suggest that DNA-encoded nanochemistry is a viable route to synthesize customized nanostructures which combine inorganic, biologic, and molecular components in defined stoichiometries. In practice, however, it has been difficult to realize the full potential of this vision, because producing arbitrary nanoparticles with a defined DNA valency remains a significant challenge.

In general, DNA valency-defined nanoparticles cannot be obtained directly from chemical reactions, because the effectively isotropic reactivity of the nanoparticle surface leads DNA attachment to proceed randomly, generating a mixture of products. To obtain a specific product, works to date have focused either on exerting regioselective control over DNA attachment using specialized DNA structures or, more generally, on isolating a desired species from a mixture using purification techniques like gel electrophoresis or anion-exchange chromatography. Unfortunately, these approaches seem to be effective only for spherical nanoparticles with diameters up to approximately 30-40 nm. This size limit excludes many nanomaterials with unique functionality, including plasmonically active and highly scattering metallic nanorods or large nanospheres, which—individually and when assembled into nanocomposites—have promising applications as optical contrast agents, photothermal therapeutics, (bio)chemical sensors, and fluorescence- or Raman-enhancing optical antennas.

A fundamental problem with existing approaches for obtaining valency-defined nanoparticles is that their selectivity is based on physical characteristics such as charge and size, which are reflective of the nanoparticle as a whole, rather than the attached DNA specifically. As a result, the DNA features register as different shades of a continuous analog signal, and as nanoparticle size increases or surface chemistry changes, they become a nearly indistinguishable perturbation.

### BRIEF SUMMARY

In some embodiments, a method for sorting nanoparticles or molecules may be provided. The method may include providing a plurality of nanoparticles or molecules, where each nanoparticle or molecule may include one or more keyword sequences (e.g., such as a 5-nt to 20-nt sequence). Each keyword sequence may be appended onto a DNA sequence that may be attached to the nanoparticle or molecule. The method may include allowing the keyword sequences on each nanoparticle or molecule to bind to a capture sequence coupled to a solid support substrate. Each capture sequence may be a reverse complement of the keyword sequence. The method may include releasing the nanoparticle or molecule on the solid support substrate based on a mobile phase strength of a mobile phase passing over the solid support substrate.

In some embodiments, at least one of the plurality of nanoparticles or molecules may include a plurality of keyword sequences coupled to the nanoparticle or molecule. In some embodiments, the plurality of keyword sequences are the same keyword sequence. In some embodiments, at least one of the plurality of keyword sequences is different from another of the plurality of keyword sequences. In some embodiments, each keyword sequence of the plurality of keyword sequences is different. In some embodiments, each nanoparticle or molecule comprising a plurality of different keyword sequences coupled to the nanoparticle or molecule. In some embodiments, each different keyword sequence is coupled to one or more DNA sequences. In some embodiments, each different keyword sequence is coupled to a plurality of DNA sequences. In some embodiments, the at least one of the plurality of nanoparticles or molecules comprises a plurality of different molecules, each molecule coupled to a different keyword sequence.

In some embodiments, the nanoparticle or molecule may be a gold nanoparticle, a silver nanoparticle, an iron oxide nanoparticle, a semiconducting nanocrystal, a gold nanorod, a small molecule, a ligand, a protein, or an antibody.

In some embodiments, the solid support substrate may be an exclusion chromatography resin. In some embodiments, the capture sequence may be grafted to the exclusion chromatography resin via carbonyldiimidazole coupling chemistry. In some embodiments, the solid support substrate may be a monolithic support.

In some embodiments, the nanoparticles or molecules are at least partially coated with a coating material, such as a polyethylene glycol (PEG). In some embodiments, at least one additional material is bound to the nanoparticle, such as an additional nanoparticle or a biomolecule.

In some embodiments, the method may include determining at least one valency of the plurality of nanoparticles or molecules based on a retention time, volume of mobile phase utilized, or a combination thereof. In some embodiments, the method may include determining an identity of the molecule based on a retention time, volume of mobile phase utilized, or a combination thereof. In some embodiments, the method may include injecting a first buffer into a column, then injecting a sample containing the plurality of nanoparticles into the column. In some embodiments, the method may include collecting fractions, pooling the fractions, and concentrating the pooled fractions.

In some embodiments, the method may include determining a UV-Vis spectra of a sample containing a released bound nanoparticle. In some embodiments, the method may include determining an optical density of a sample containing a released bound nanoparticle. In some embodiments, the method may include determining a fluorescence of a sample containing a released bound nanoparticle. In some embodiments, the method may include determining a refractive index of a sample containing a released bound nanoparticle.

In some embodiments, the mobile phase strength may be modulated in a linear manner. In some embodiments, modulating the mobile phase strength may include decreasing the mobile phase concentration of NaCl in a linear gradient. In some embodiments, the mobile phase strength may be modulated in a non-linear manner.

In some embodiments, the method may include washing the solid structure with a solvent after releasing the bound nanoparticles.

In some embodiments, the method may include collecting the plurality of nanoparticles or molecules after release. In some embodiments, the method may include drying and/or purifying the collected plurality of nanoparticles or molecules, where each nanoparticle or molecule is a colored, magnetic, or photoluminescent nanoparticle, a small molecule, or a biomolecule, and each nanoparticle or molecule bears a single bio-active molecule or reactive group.

In some embodiments, a system DNA valency sorting chromatography may be provided. The system may include a column packed with a solid support substrate as disclosed herein and a solvent, where the solid support substrate may be coupled to a plurality of capture sequences. The system may include a plurality of nanoparticles or molecules as disclosed herein within the column, where at least one keyword sequence may be appended to a DNA sequence and is attached to each nanoparticle, where each keyword sequence may be a complement to the capture sequence.

In some embodiments, a composition of matter may be provided. The composition of matter may include a plurality of nanoparticles or molecules coupled together, each nanoparticle being coupled to at least one other of the plurality of nanoparticles via a nucleotide connection. Each nucleotide connection may independently comprise: (1) a keyword sequence appended to a DNA sequence attached to one of the nanoparticles being coupled; and (2) a capture sequence appended to a DNA sequence attached to the other of the nanoparticles being coupled, the capture sequence being a reverse complement of the keyword sequence. In some embodiments, the composition of matter includes at least three nanoparticles or molecules coupled together.

### BRIEF DESCRIPTION OF DRAWINGS

FIGS. 1A-1D are graphical representations of idealized steps for sorting α-tagged nanoparticles by DNA valency, including a introductory stage (1A), after washing (1B), after eluting at a first concentration of a mobile phase (1C), and after eluting at a second concentration of the mobile phase (1D).

FIG. 2 is a flowchart of a method.

FIG. 3 is a simplified illustration of a nanoparticle or molecule coupled to a keyword sequence.

FIG. 4 is a simplified illustration of a system.

FIGS. 5A and 5B are simplified illustrations of compositions of matter comprising two (5A) or three (5B) nanoparticles or molecules (5A).

FIG. 6 is a graph showing the separation of 5 nm PEG-coated S1-AuNPs by DNA valency sorting.

FIG. 7 is a graphical illustration of a nanoparticle or molecule with an attached keyword sequence hybridized to a second DNA strand.

FIG. 8 is a graphical illustration of a dimer by unmediated handshake bonding between monovalent nanoparticle or molecule and a polyvalent nanoparticle or molecule.

FIG. 9 is a graphical illustration of a dimer by template directed bonding between two identical monovalent nanoparticles or molecules.

FIG. 10 is a graphical illustration of a dimer antenna created by clamping two distinct monovalent nanoparticles or molecules through a complementary intermediate strand.

FIG. 11 is a Histogram of TIRF emission intensities from single molecules of Cy®3 associated with dsDNA control (n=298) or 80 nm dimers (n=148).

FIG. 12 is a depiction of certain chemical structures referenced in Table 1.

FIG. 13A shows chromatograms of 10-60 nm S1-AuNPs coated with PEG 2. Unlabeled and monovalent peaks are indicated by filled and open arrows, respectively.

FIGS. 13B and 13C are graphs showing Peak width (13B) and retention volume (13C) of unlabeled and monovalent peaks from chromatograms in FIG. 13A versus hydrodynamic diameter measured by DLS. arb. u.=arbitrary units. Solid lines are a guide to the eye.

FIGS. 14A-14C are graphs illustrating the effect of resins with different size exclusion properties on the retention of 20 nm unlabeled (filled markers) or monovalent S1-AuNPs (open markers). Chromatograms were acquired on miniature (1.5 mL) columns packed with α′-modified resins: Sephacryl® S-400 (14A), Sepahcryl® S-300 (14B), and Sepahcryl® S-200 (14C). arb. u.=arbitrary units.

FIG. 15A shows chromatograms of 20 nm S1-AuNPs coated with indicated PEG ligands. Unlabeled and monovalent peaks are indicated by filled and open arrows, respectively.

FIGS. 15B and 15C are graphs showing retention volume of unlabeled (15B) and monovalent peaks (15C) from chromatograms in FIG. 15A versus PEG MW. arb. u.=arbitrary units. Solid lines are a guide to the eye.

FIG. 16A are graphs showing the effect of PEG ligands on exclusion and evidence for steric barrier to affinity interaction. The left graph shows average size exclusion distribution coefficient K_{SEC} of unlabeled peak versus hydrodynamic diameter for 10-60 nm AuNPs coated with PEG 2 and 20 nm AuNPs coated with indicated PEG ligands, while the right graph shows apparent retention factor (k′ₐₚₚ) of monovalent peak versus hydrodynamic diameter for 10-60 nm S1-AuNPs coated with PEG 2 and 20 nm S1-AuNPs coated with indicated PEG ligands. Dashed lines are linear fit to the 10-60 nm data.

FIG. 16B is an illustration of the structure of the d(S1) DNA complex used to extend the keyword sequence α from the nanoparticle surface.

FIG. 16C is a representative chromatogram of S1- and d(S1)-AuNPs coated with PEG 5 (zoom of monovalent peak), showing qualitative shift in retention volume.

FIG. 16D is a plot of ratio k′ₐₚₚ for d(S1)-AuNPs to k′ₐₚₚ for S1-AuNPs versus PEG thickness.

FIG. 17A is a chromatograph showing resolution of 60 nm S1-AuNPs as a result of varying [NaCl] in buffer A at constant flow rate (0.4 ml/min).

FIG. 17B is a graph showing retention volume from chromatographs in FIG. 17A versus [NaCl].

FIG. 17C is a chromatograph showing resolution of 60 nm S1-AuNPs as a result of varying flow rate at constant [NaCl] in buffer A (250 mM).

FIG. 17D is a graph showing peak width from chromatograms in FIG. 17C versus flow rate.

FIG. 18A are graphs showing a breakthrough analysis of the dynamic binding capacity of two DNA valency sorting monoliths, a 0.1 mL monolith disks modified with α′-T₁₅ (18A) or α′-T₅ (18B) DNA.

FIGS. 19A-19D are chromatograms showing preliminary DNA valency sorting of 5 nm S1-AuNPs on various columns, including on the conventional column prepared from Sephacryl® S-1000 low pressure resin (diameter: 10 mm, height: 185 mm) (19A), on the α′-T₁₅ monolith (19B), on the α′-T₅ monolith (19C), and on the α′-T₅ monolith (19C) at varying flow rates (19D). For clarity all chromatograms have been normalized to the maximum absorbance.

FIG. 20A is a graph showing experimentally measured (HETP)_{LGE} versus linear mobile phase velocity u for the three indicated columns. Data points are mean±standard deviations of at least four separate experiments. Solid lines are linear fits to the data points.

FIG. 20B is a graph showing Calculated gradient volume V_{g} required to obtain equivalent resolution on the α′-T₅ monolith, compared to the packed bed column, as a function of flow rate. Different initial concentrations of NaCl in the start buffer (in M) are indicated by the curves.

FIG. 20C is a graph showing calculated gradient time, t_{g}=V_{g}/f, for equivalent resolution separation on the α′-T₅ monolith as a function of flow rate. Concentration of NaCl in the start buffer (in M) is indicated by each curve.

FIG. 20D is a graph showing experimental isoresolution separations of 5 nm S1-AuNPs on the α′-T₁₅ (V_{g}=1.15 mL, f=0.02 mL/min, [NaCl]₀=0.200 M) and α′-T₅ (V_{g}=1.79 mL, f=0.02 mL/min, [NaCl]₀=0.100 M) monoliths. The equivalent separation of the packed bed column is shown for comparison. For clarity, the volume was adjusted to zero at the retention volume of the monovalent peak (arrow), then normalized by the total volume of the program.

FIG. 21A shows chromatograms of S1-AuNPs stabilized with a carboxy-termined PEG ligand (MW 1000; COOH-PEG₁₀₀₀) obtained on the α-T₅ monolith, analyzed using a method with [NaCl]=125 mM, V_{g}=2 mL, f=0.02 mL/min. Chromatograms are normalized the height of the retained peaks for ease of visualization.

FIG. 21B is a graph showing average resolution of the monovalent peak for chromatograms on the α-T₅ monolith shown in FIG. 21A compared to the optimal methods on the packed bed column.

FIG. 21C is a graph showing analysis time of methods on α-T₅ monolith shown in FIG. 21A compared to optimal methods on the packed bed column.

FIG. 22A is an illustration of S2′-S2-NPs.

FIG. 22B is a chromatogram showing separation of 5 nm PEG-coated S2′-S2-AuNPs, compared to S1-AuNPs of the same diameter.

FIG. 22C are chromatograms of 5-80 nm PEG coated S1-AuNPs. For 5-20 nm NPs, one method was used, and for 40-80 nm NPs, [NaCl] was increased to 150, 200, and 280 mM respectively, and the flow rate was reduced to 0.1 mL/min.

FIG. 22D is a chromatograph from sorting 40×92 nm PEG-coated S1-tagged gold nanorods (detected at 546 nm); initial [NaCl] was 280 mM and flow rate was 0.1 mL/min.

FIG. 22E is a chromatogram (detected at 254 nm) from sorting S1-tagged streptavidin-coated quantum dots emitting at 565 nm; initial [NaCl] was 100 mM and flow rate was 0.4 mL/min.

FIG. 22F is a chromatogram (detected at 436 nm) from sorting S1-tagged 20 nm streptavidin-coated iron (II, III) oxide nanocrystals emitting at 565 nm; initial [NaCl] was 250 mM and flow rate was 0.1 mL/min.

### DETAILED DESCRIPTION

To overcome the above-described limitations, the disclosed DNA valency sorting chromatography may be utilized. This technique exploits the highly selective and programmable association of complementary DNA sequences to separate nanoparticles bearing a specified valency of DNA molecules.

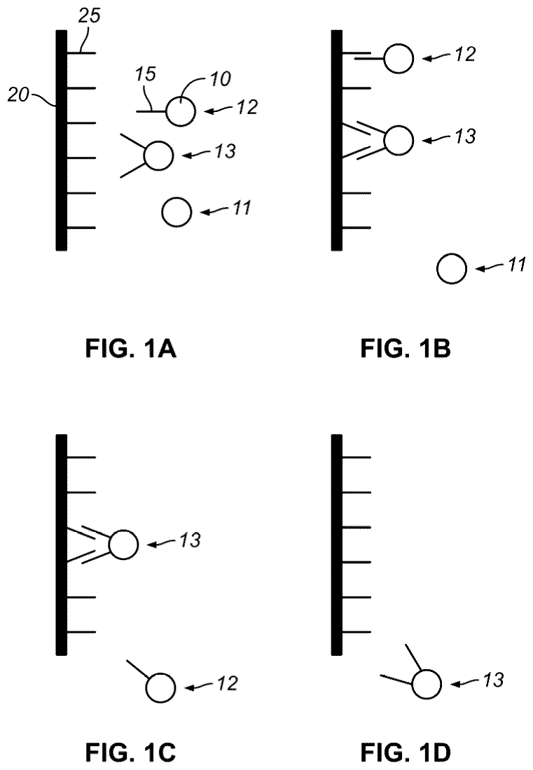

Conceptually, the specific interaction between Watson-Crick base pairs and the cooperativity inherent in multivalent interactions provide a unique, digital read-out for the number of DNA strands on a nanoparticle that should be independent of the characteristics of the nanoparticle itself. In practice, and in reference to FIGS. 1A-1D, a keyword sequence 15 (α) is included in the DNA attached to a nanoparticle 10 (untagged particle 11, univalent particle 12, and divalent particle 13 are shown), and its reverse complement (α′, the capture sequence 25) is bound/fixed to a stationary phase 20. See FIGS. 1A-1B. Essentially, the nanoparticle's DNA valency becomes “encoded” in its affinity for the immobilized capture sequence. A column packed with the stationary phase then acts as a “decoder,” first recognizing α-tagged nanoparticles with high specificity (see FIG. 1B, where untagged particles 10 are washed out, while univalent particle 12 and divalent particle 13 are bound to the stationary phase). Subsequently, as mobile phase conditions are gradually changed, sorting them by their affinity into nanoparticles with defined DNA valency (see FIG. 1C, where at a first concentration of, e.g., NaCl in the mobile phase, the univalent particles 12 are eluted. In FIG. 1D, as the concentration changes to a second, further reduced concentration of NaCl, the divalent particles 13 are then eluted).

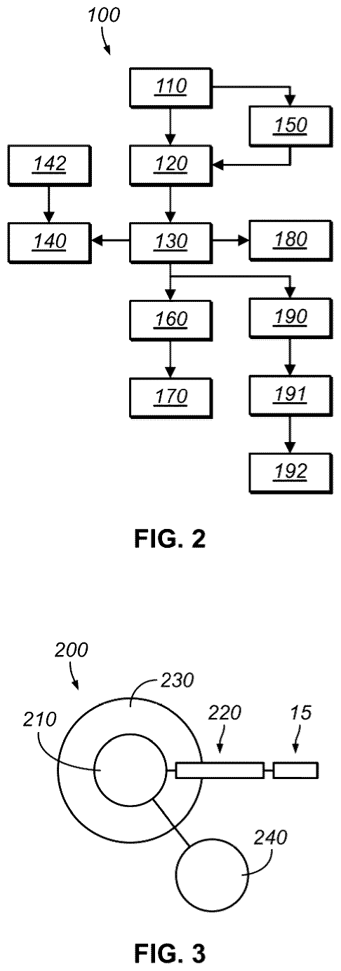

Thus, in some embodiments, a method for sorting nanoparticles or molecules may be provided. Referring to FIGS. 2 and 3, the method 100 may include providing 110 a plurality of nanoparticles or molecules 200. The nanoparticle or molecule may include any appropriate nanoparticle or molecule 210 as a base. In some embodiments, the nanoparticle or molecule may be a gold nanoparticle, a silver nanoparticle, an iron oxide nanoparticle, a semiconducting nanocrystal, a gold nanorod, a small molecule, a ligand, a protein, or an antibody. In some embodiments, each nanoparticle or molecule may be a colored, magnetic, and/or photoluminescent nanoparticle, a small molecule (e.g., ≤1000 Daltons), or a biomolecule.

Each nanoparticle or molecule may include a keyword sequence 15. The keyword sequence may be coupled to the base nanoparticle or molecule 210. In some embodiments, the keyword sequence may be a 5-nt to 50-nt sequence. In some embodiments, the keyword sequence may be a 5-nt to 40-nt sequence. In some embodiments, the keyword sequence may be a 5-nt to 30-nt sequence. In some embodiments, the keyword sequence may be a 5-nt to 20-nt sequence. In some embodiments, the keyword sequence may be a 5-nt to 15-nt sequence. In some embodiments, the keyword sequence may be an 8-nt to 12-nt sequence.

In some embodiments, at least one nanoparticle or molecule may include a plurality of keyword sequences. In some embodiments, each nanoparticle or molecule may include a plurality of keyword sequences. In some embodiments, each of the plurality of keyword sequences is the same. In some embodiments, each of the plurality of keyword sequences may be different.

Each keyword sequence may be appended onto a DNA sequence 220 attached or coupled to the nanoparticle or molecule. The DNA sequence may be any DNA sequence that is not part of the keyword(s). The DNA sequence is not particularly limited in length, although in some embodiments, it may be 50 nucleotides in length or shorter. In some embodiments, the DNA sequence may be 1-40 nucleotides in length. In some embodiments, the DNA sequence may be 1-30 nucleotides in length. In some embodiments, the DNA sequence may be 5-20 nucleotides in length. In some embodiments, repeating nucleotides sequences (e.g., 5-20 repeating thymines, adenines, cytosines, or guanines) may be used.

In some embodiments, the plurality of nanoparticles or molecules comprises a plurality of different molecules, each molecule coupled to at least one different keyword sequence. In some embodiments, each nanoparticle or molecule may include a plurality of different keyword sequences coupled to the nanoparticle or molecule. In some embodiments, the plurality of nanoparticles or molecules comprises a plurality of different particles, each molecule coupled to at least one different keyword sequence. In some embodiments, each different keyword sequence may be coupled to one or more DNA sequences. In some embodiments, each different keyword sequence may be coupled to a plurality of DNA sequences.

In some embodiments, the nanoparticles or molecules may be at least partially coated with a coating material 230. In some embodiments, the coating may be a polymer. In some embodiments, the coating may include, e.g., a polyethylene glycol (PEG).

In some embodiments, each nanoparticle or molecule may be bound to another nanoparticle or molecule 240. In some embodiments, the molecule 240 is a biomolecule. In some embodiments, each nanoparticle or molecule may bear a single bio-active molecule or reactive group.

The method may include allowing 120 the keyword sequences on each nanoparticle or molecule to bind to a capture sequence (such as capture sequence 25 in FIG. 1A) coupled to a solid support substrate (such as stationary phase 20 in FIG. 1A). Each capture sequence is a reverse complement of one of the keyword sequences.

In some embodiments, the solid support substrate may be an exclusion chromatography resin. In some embodiments, the capture sequence may be grafted to the exclusion chromatography resin via, e.g., carbonyldiimidazole coupling chemistry.

In some embodiments, the solid support substrate is a monolithic support.

The method may include releasing 130 the nanoparticle or molecule on the solid support substrate based on a mobile phase strength of a mobile phase passing over the solid support substrate.

In some embodiments, the mobile phase strength may be modulated in a linear manner. For example, in some embodiments, modulating the mobile phase strength may include decreasing the mobile phase concentration of NaCl in a linear gradient. In some embodiments, the mobile phase strength is modulated in a non-linear manner.

In some embodiments, higher valency particles or molecules release from the solid support substrate at lower concentrations of a material in the stationary phase than lower valency particles. In some embodiments, the reverse is true; that is, higher valency particles or molecules release from the solid support substrate at higher concentrations of a material in the stationary phase than lower valency particles.

In some embodiments, the method may include determining 140 a characteristic and/or an identify of the nanoparticle or molecule based on a retention time, volume of mobile phase utilized, or a combination thereof. For example, by previously creating 142 various calibration curves, one or more processors may be configured by instructions on a non-transitory computer readable storage medium to determine (or estimate) a valency based on retention time and/or volume of mobile phase utilized. Similarly, with calibration curves, an identify of the nanoparticle or molecule based on based on a retention time, volume of mobile phase utilized, or a combination thereof can be determined. In some cases, rather than a calibration curve, a model or equation (which may be, e.g., based on empirical data) may be used to determine valency and/or identify of the nanoparticle or molecule. For example, if the concentrations of a material in a mobile phase at which a particular valency is eluted are known, and the gradient for that material is known, the time at which the particular valency should be eluted can be calculated.

In some embodiments, the method may include injecting 150 material into a column. For example, in some embodiments, the method may include injecting a first buffer into a column, then injecting a sample containing the plurality of nanoparticles into the column.

In some embodiments, the method may include collecting 160 a sample. In some embodiments, collecting a sample may include collecting fractions. The method may also include pooling the fractions. The method may also include concentrating the pooled fractions.

In some embodiments, the method may include determining 170 a characteristic of a sample containing a released bound nanoparticle. In some embodiments, determining a characteristic may include determining a UV-Vis spectra of a sample containing a released bound nanoparticle. In some embodiments, determining a characteristic may include determining an optical density of a sample containing a released bound nanoparticle. In some embodiments, determining a characteristic may include determining a fluorescence of a sample containing a released bound nanoparticle. In some embodiments, determining a characteristic may include determining a refractive index of a sample containing a released bound nanoparticle.

In some embodiments, the method may include washing 180 the solid support structure with a solvent after releasing the bound nanoparticles.

In some embodiments, the method may include collecting 190 some or all of the plurality of nanoparticles or molecules after release. In some embodiments, the method may include drying and/or purifying 191 the collected plurality of nanoparticles or molecules. In some embodiments.

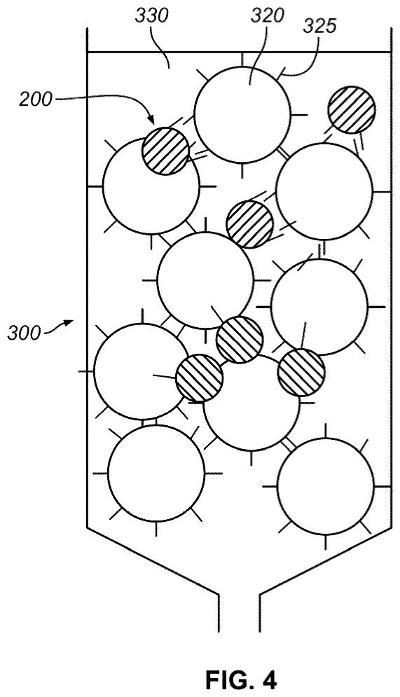

In some embodiments, a system for DNA valency sorting chromatography may be provided, Referring to FIG. 4, the system may include a column 300 packed with a solid support substrate 320 and a solvent 330, where the solid support substrate may be coupled to a plurality of capture sequences 325. The system may include a plurality of nanoparticles or molecules 200 as disclosed herein within the column, where at least one keyword sequence appended to a DNA sequence is attached to each nanoparticle or molecule, each keyword sequence being a complement to the capture sequence.

As will be understood by those of skill in the art, in some cases, the solvent 330 may include a packing solvent, such as water, or water and a surfactant. As will be understood by those of skill in the art, in some cases, the nanoparticles or molecules 200 and solvent 330 may form the mobile phase within the column.

Considering the binding between the keyword and its reverse complement, it is possible to use the nanoparticle or molecule structures described here to form complex compositions of matter comprising multiple nanoparticles or molecules.

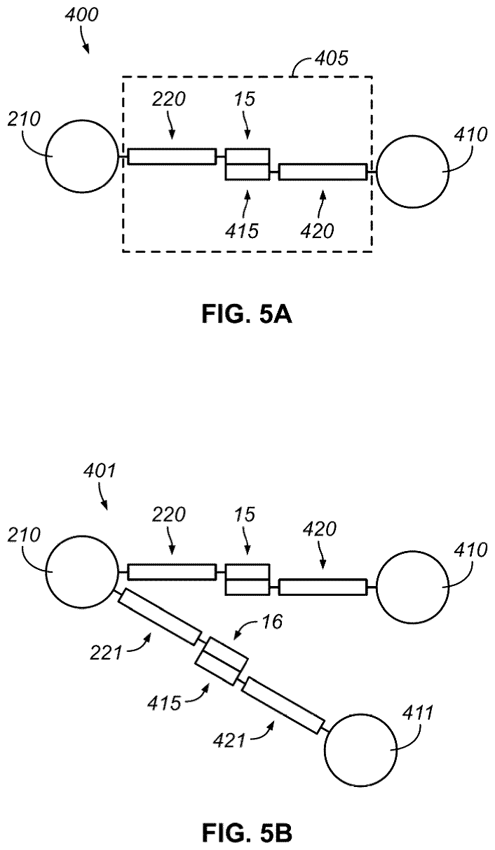

Referring to FIG. 5A, in some embodiments, a composition of matter 400 may be provided, comprising a plurality of nanoparticles coupled together. Each nanoparticle is coupled to at least one other of the plurality of nanoparticles (here, because each nanoparticle 210 is univalent, nanoparticle 210 is only coupled to nanoparticle 410, and vice-versa) via a nucleotide connection 405.

Each nucleotide connection may, independently, include a keyword sequence 15 (which may be appended to a DNA sequence 220) attached to one of the nanoparticles being coupled, and a capture sequence 415 (which may be appended to a DNA sequence 420) attached to the other of the nanoparticles being coupled, the capture sequence being a reverse complement of the keyword sequence. It does not matter which nanoparticle the keyword sequence or capture sequence is coupled to, so long as the nucleotide connection contains those elements. The keyword sequence will bind/adhere to the capture sequence, coupling the two nanoparticles together.

With multi-particle or multi-molecule compositions, in some embodiments, each particle or molecule may be the same. In some embodiments, each particle or molecule may be different. For example, as seen in FIG. 5A, in some embodiments, nanoparticles or molecules 210 and 410 may be the same. In some embodiments, nanoparticle or molecule 210 may be different from nanoparticle or molecule 410.

One of skill in the art will recognize that, as valency increases, the complexity of compositions of matter that can be formed increases. In some embodiments, the composition of matter may include at least three nanoparticles or molecules.

For example, as seen in FIG. 5B, a three-nanoparticle or molecule composition can be created, provided at least one nanoparticle or molecule is divalent (here, nanoparticle or molecule 210). The two others (here, nanoparticles or molecules 410 and 411) are shown as being univalent. A three-particle system can be also created if all three are divalent, and they are coupled with each other to form a triangular configuration.

As seen in FIG. 5B, the first nanoparticle or molecule 210 may be coupled to a second nanoparticle or molecule 410 in a similar fashion to that seen in FIG. 5A. Because the first nanoparticle or molecule 210 is divalent, a third nanoparticle 411 can also be coupled to the first nanoparticle or molecule through a second nucleotide connection. The second nucleotide connection may include, e.g., a second keyword sequence 16 (which may be appended to a DNA sequence 221) attached to one of the nanoparticles being coupled, and a capture sequence 416 (which may be appended to a DNA sequence 421) attached to the other of the nanoparticles.

In some embodiments of the composition of matter, every keyword sequence attached to a given nanoparticle or molecule is identical. In some embodiments, at least one keyword sequence is different. In some embodiments, at least one nanoparticle or molecule is divalent. In some embodiments, at least one nanoparticle or molecule is trivalent. In some embodiments, at least one nanoparticle or molecule is tetravalent. In some embodiments, at least one nanoparticle or molecule is pentavalent. In some embodiments, at least one nanoparticle or molecule is hexavalent. In some embodiments, at least two nanoparticles or molecules are multivalent.

### Example 1

Gold nanoparticles (AuNPs) were used as a representative nanomaterial because of their high extinction coefficients, ease of chemical modification, and commercial availability in a range of sizes. In this experimental implementation, the keyword α was selected as the ten nucleotide (nt) sequence 5′-CTTGTGTCTA-3′ [SEQ ID NO. 1]. It was included at the 5′-end of a longer DNA strand, denoted S1 [SEQ ID NO. 3] which was attached to the AuNP surface at its 3′-end through two sequential alkyl thiol groups. The capture resin of the valency sorting column (α′-resin) was prepared by covalently immobilizing a DNA strand, composed of the capture sequence α′ (5′-TAGACACAAG-3′) [SEQ ID NO. 2] and a 15-nt poly-T spacer, onto a low pressure gel filtration chromatography support.

The α′-resin was prepared by first converting hydroxyl groups on Sephacryl-1000 to activated carbamates by carbonyldiimidazole (CDI). Sephacryl® S-1000 (20 mL settled resin) was gradually washed into dry acetone by vacuum filtration over a coarse fritted glass filter. The washed resin was diluted to a 50% slurry in acetone and transferred to a glass flask. Solid CDI (1.66 g) was added slowly to the slurry while stirring with a glass rod. The flask was covered to prevent evaporation and gently swirled every 10 minutes for 60 minutes total. The CDI-Sephacryl® was recovered by vacuum filtration and washed multiple times with acetone, then finally diluted to a 50% slurry in acetone and stored in a glass bottle at 4° C. until use. The concentration of CDI groups on the resin was measured following Ngo, and typical values were 26 μmol CDI per mL settled resin. Next, oligonucleotide α′-T₁₅-am was mixed with CDI-Sephacryl®. To prepare sufficient α′-resin to pack a chromatography column, multiple sequential small-scale coupling reactions were performed and the unreacted α′-T₁₅-am was recovered after each one. In a typical small-scale reaction, CDI-Sephacryl® (1.4 mL; 50% slurry in acetone) was washed by vacuum filtration over a fritted glass filter multiple times with ice-cold water. The settled resin was then combined with α′-T₁₅-am (0.38 mL; 3.8 mM in water) and 0.5 M sodium borate, pH 8.5 (0.28 mL) to obtain a mixture with a final composition of 50% slurry, 1 mM α′-T₁₅-am, and 0.1 M sodium borate. The reaction was mixed by gentle rotation for 12-24 hours at room temperature. Then the α′-resin was recovered by vacuum filtration and washed with 5 mL each of water, 1 M NaCl, and water. The unreacted α′-T₁₅-am was recovered from the washes by ethanol precipitation. This procedure was repeated until ˜20 mL of α′-resin was obtained. The α′-resin was stored as a 50% slurry in water at 4° C. until packing.

The concentration of α′ on the resin was measured by a fluorescence staining assay using ssDNA-binding dye Sybr™ Gold stain. α′-resin was serially diluted into 1× TE to prepare seven solutions with 0.195-12.5% slurry. The diluted slurries were mixed with an equivalent volume of 2× Sybr™ Gold stain solution in 1× TE. Similar mixtures of Sybr™ Gold stain and unmodified Sephacryl® S-1000 were used as a blank. Stained slurries were transferred to the wells of a black 384-well microplate and the plate was centrifuged (3 krpm, 10 min) to settle the resin. Fluorescence was measured (excitation 495 nm, emission 550 nm) in each well using a microplate reader (Molecular Devices, M5e). Measured fluorescence was corrected for background by subtracting the appropriate blank. The fluorescence intensity was converted to [α′] using a linear calibration curve generated from known concentrations of unmodified oligonucleotide α′-T₁₅ in the same buffer and similarly stained with Sybr™ Gold stain. Linear fit to the [α′] versus slurry % was used to extrapolate to the [α′] at 100% slurry. The [α′] for the resin used here was 9 nmol/mL settled resin.

A central premise of DNA valency sorting is that nanoparticles bearing different numbers of α will exhibit distinct affinities for the α′-resin. To confirm that this condition was met, the dissociation constant (K_{d}) was measured between α′-resin and 5 nm diameter AuNPs bearing one, two, or three copies of S1. The 5 nm AuNPs of defined S1 valency were purified by agarose gel electrophoresis and subsequently coated with a thiol-modified poly (ethylene glycol) (PEG) ligand. The K_{d} was measured in a batchwise equilibrium binding assay by monitoring the amount of S1-AuNPs remaining in the supernatant after exposure to various amounts of α′-resin in a buffer containing 150 mM [NaCl]. Fitting the experimental data revealed that mono-, di-, and tri-valent S1-AuNPs had K_{d} values of 0.32±0.23 μM, 0.042±200.018 μM, and 0.0134±0.0099 μM, respectively. As expected, the S1-AuNPs bound to the resin, and—critically—their affinity increased with α valency.

AuNPs were stabilized with BSPP and concentrated ˜25-fold following existing procedures. Typical concentrations of 5, 10, 20, 40, 60, and 80 nm BSPP-AuNPs were 3000, 300, 30, 3, 1, and 0.3 nM, respectively. Unless otherwise noted, to centrifuge 10, 20, 40, 60 and 80 nm AuNPs, 1 mL of solution was centrifuged for 30 min at 10, 4.5, 2, 1, 0.6 krcf. To prepare S1-AuNPs, internal disulfides in S1-th2 were reduced by diluting it to 1-5 μM in 1 mM BSPP and incubating for at least 1 hour. Reduced S1-th2 was mixed with BSPP-AuNPs, NaCl, and 0.5× TE buffer and incubated for ˜18 hours. Typical reaction conditions for different AuNP diameters were as follows. 5 nm: 0.3-2 equiv. S1, 50 mM NaCl; 10 nm: 2-10 equiv. S1, 50 mM NaCl; 20-60 nm: 2-30 equiv. S1, 20 mM NaCl; 80 nm: 20-500 equiv. S1, 13 mM NaCl, 0.03% SDS. After labelling with S1, the AuNPs (except for 5 nm diameter) were washed once with 1 mM BSPP by centrifugation, then redispersed in 1× TE prior to PEG coating. To coat with PEG, stock solutions of various PEG molecules were prepared as follows. mPEG₃₅₀-SH was diluted to 1/50 in water (˜60 mM). COOH-PEG₆-C₁₁-SH was diluted 1/100 into 1× TE (˜20 mM). COOH-PEG₁₀₀₀-SH and COOH-PEG₂₀₀₀-SH were prepared as 5 mM solutions in 1× TE then diluted 1/10 into 5 mM BSPP and incubated for at least 1 hour. ⅕ volume of mPEG₃₅₀-SH, COOH-PEG₆-C₁₁-SH, COOH-PEG₁₀₀₀-SH, and COOH-PEG₂₀₀₀-SH solutions were added to 5-10 nm, 20-40 nm, 60 nm, and 80 nm DNA-tagged AuNPs, respectively. The mixtures were incubated at least 30 minutes prior to DNA valency sorting.

Measurement of the dissociation constant between 5 nm S1-AuNPs and α′-resin. 5 nm S1-AuNPs were prepared as described above using 0.7 equivalents of S1-th2, but not coated with PEG. Individual valencies were purified by separating the mixture on a 3% agarose gel at 7 V/cm for 1.5 to 2 hours. Bands corresponding to S1-AuNPs with one, two, or three copies of S1 were extracted with a scalpel and AuNPs were recovered from the gel slice by crushing it with a teflon pestle and immersing it in 0.5× TBE. After 24 hours of extraction, the liquid containing the purified S1-AuNPs was separated from the crushed gel by vacuum filtration over a 0.22 μm PES filter. The recovered S1-AuNPs were concentrated to 1-3 μM with a 30 kDa MWCO centrifugal filter (Amicon Ultra, Millipore). For a total 0.9 nmol input S1-AuNPs, the amount of purified S1-AuNPs for each valency was ˜0.1 to 0.15 nmol. Immediately prior to performing the equilibrium binding experiment, purified S1-AuNPs were incubated with 100-fold molar excess of mPEG₃₅₀-SH.

To measure the dissociation constant, mono-, di- and tri-valent S1-AuNPs were separately incubated with different amounts of α′-resin. Resin slurries were prepared in water by serially diluting a 70% slurry of α′-resin ([α′]=13.6 nmol/mL) with a 70% slurry of unmodified Sephacryl® S-1000. Ten slurries were obtained containing 0.02-100% α′-resin. PEG-coated purified S1-AuNPs (˜200 nM) were prepared in buffer containing 2× TE, 300 mM NaCl, and 0.02% SDS. Aliquots of the S1-AuNP solutions were mixed in equal volume with each prepared resin slurry. The final composition of the solutions was 100 nM S1-AuNPs, 1× TE, 150 mM NaCl, 0.01% SDS, and 35% slurry of resin, and the final [α′] varied from 0.0012 to 4.8 μM. The mixtures were rotated gently for 2-4 hours at room temperature, the resin was settled by gravity, and a UV-Vis spectrum was measured of the supernatant. Two measurements were performed for each mixture and three separate replicates of the assay were performed for each S1-AuNP valency.

To process the data, all UV-Vis spectra were baseline subtracted at 850 nm. Since fine resin particles may remain in the supernatant and contribute a variable scattering signal to the spectrum, the following procedure was used to remove the scattered signal. A normalized scattering spectrum, S(λ), was generated by measuring the spectrum of a solution of resin particles and dividing by OD at 400 nm. Using this normalized spectrum, the sample-specific scattering spectrum was approximated by f(λ)=S(λ)×C, where Cis given by (OD₄₀₀-OD₄₅₀)/(1−S(450)). Finally, f(λ) was subtracted from the baseline-subtracted spectrum to arrive at a corrected spectrum corresponding to the AuNP component alone. The variation of OD₅₂₀ with [α′] was used to determine the K_{d}. The dependence of OD₅₂₀ on [α′] was derived from the bimolecular equilibrium binding reaction between S1-AuNPs and α′-resin and is given by

$${OD}_{520} = A - \frac{B}{2 C} ( [ {α}^{′} ] + {K}_{d} + C - \sqrt{{( [ {α}^{′} ] + {K}_{d} + C )}^{2} - 4 C [ {α}^{′} ]} ) , \qquad ( Eq . 1 )$$

where A is the initial OD₅₂₀ of the sample, B is the OD₅₂₀ corresponding to AuNPs that can actually interact with the α′-resin, and C is the initial [S1-AuNP]. K_{d} was extracted by fitting the corrected OD₅₂₀ to this equation using non-linear least squares regression in Matlab. A, B, C, and K_{d} were fitting parameters. Each replicate experiment was fit separately using all data points. For a given S1-AuNP valency, the reported K_{d} is the average of the three experiments and the uncertainty is one standard deviation, calculated as the aggregated population standard deviation.

Characterization of reactions by gel electrophoresis. Agarose gel electrophoresis was performed in a horizontal gel running chamber. 0.5× TBE was used to pour the gel and as the running buffer. 3%, 2%, and 1% agarose (w/w) were used for 5 nm, 10-20 nm, and 40-80 nm diameter AuNPs. 30% glycerol was used as a 6× running buffer. After loading into wells, samples were typically left to sit for 1-2 minutes before running. Gels were run at a constant voltage of 7-10 V/cm for 30-90 minutes at 4° C. (for analysis of artificial molecule reactions) or room temperature (otherwise). Transmission images of gels were acquired with an AlphaImager HP gel imager (Alpha Innotech) equipped with a white light table for illumination and a 16-bit black and white CCD camera for image acquisition. Images were corrected for inhomogeneous background by dividing the image containing the gel by a background image without the gel present. Contrast of gel images was adjusted linearly for clarity. All lanes which are compared to each other were present in the same gel. Gel densitometry was used to quantify the yield of reactions where indicated. The lane of interest was cropped and saved at 8-bit depth. The pixel values were inverted by subtracting each from 256. Pixel intensity was summed across the lane to generate a lane profile, and a baseline was estimated as a straight line and subtracted. The baseline-subtracted profile was divided into segments containing observed bands and the intensity was summed across each segment to obtain the total intensity of each band. The percentage of each species was then calculated by dividing the intensity of that band by the total intensity of all bands. Uncertainty was estimated from the standard deviation of pixel intensity in a region with only background and propagated according to standard formulas.

Characterization by transmission electron microscopy (TEM). Samples for analysis were diluted with buffer (1× TE, 100 mM NaCl) to approximately OD₅₂₀ 0.5 before depositing on the grid. Carbon-coated copper grids (400 mesh; Ted Pella 01844) were glow discharged for 3 minutes in a home-built chamber. 4 μl poly-D-lysine solution (1 μg/mL in water; MW 30-70 kDa) was applied to the grid for 30 seconds, then the liquid was wicked with a piece of filter paper, and the grid was air dried. Next, 4 μl of sample was applied to the grid, and after 30 seconds it was inverted onto a piece of parafilm and incubated for 5 minutes. Afterwards, the grid was blotted with a piece of filter paper and air dried.

Images of particles were acquired from multiple different locations on the grid. We attempted to acquire unbiased statistics by scanning in a single direction across the grid and acquiring an image of each new field of view, regardless of the quantity or identity of particles encountered. For each sample, typically 20-100 images were acquired at different locations across the entire sample grid. To quantify the types of particles present in the sample, particles were manually counted in acquired images and visually classified as indicated nanostructures (i.e. monomer, dimer, etc.). Observed particles were excluded from counting if they (i) were present in large clusters (>4 NPs/cluster), (ii) embedded in debris, or (iii) could not be confidently identified as a particular species. The number of each observed nanostructure, N_{S}, was estimated to have uncertainty σ_{N}_{ₛ}=\sqrt{Nₛ}, accounting for the discrete nature of the counting procedure. For characterization of dimer reactions, the number of NPs in each detected nanostructure (Nₚ) is reported and used for calculating yield. Nₚ was obtained by first counting N_{S}, then converting to Nₚ=N_{S}×n, where n is the number of NPs in a species (monomer: n=1, dimer: n=2, and so forth). The uncertainty in Nₚ was propagated as σ_{N}_{ₚ}=n×\sqrt{Nₛ}.

The DNA valency sorting procedure was tested by analyzing an input mixture of 5 nm AuNPs, including some untagged AuNPs, and mono-, di-, tri-, and tetra-valent S1-AuNPs stabilized by PEG on a column packed with α′-resin.

The column was prepared by packing α′-resin into a Tricorn 10/200 column tube (GE Healthcare), generally following the manufacturer's instructions. Water or water +0.1% Tween-20 were used as packing solvents. Typically, 16-18 mL settled α′-resin was suspended to a 70% slurry in packing solvent and degassed under vacuum for 20 minutes. The degassed slurry was poured into the column equipped with a 100 mm packing adapter and packed at a flow rate of 0.5-0.6 mL/min for 1 hour. The packing adapter was removed and replaced with a flow adapter and coarse inlet filter. The column was further packed at 0.5-0.6 mL/min until it reached an equilibrium height. The two columns had dimensions 10×203 mm and 10×210 mm, corresponding to geometrical volumes of 15.9 and 16.5 mL, respectively. Column efficiency (N) and peak asymmetry (A_{S}) was measured by applying a pulse of 0.8 M NaCl and eluting with 0.4 M NaCl at a flow rate of 0.2 mL/min. Packing was deemed acceptable if N>5000 plates/m and A_{S} was between 0.8 and 1.2. All chromatography was performed with an AktaPrime Plus (GE Healthcare) system. The elutant was monitored by a UV detector (2 mm flow cell) equipped with a 546 nm filter, followed by a conductance meter. Chromatograms were recorded with Prime View software (version 1.0).

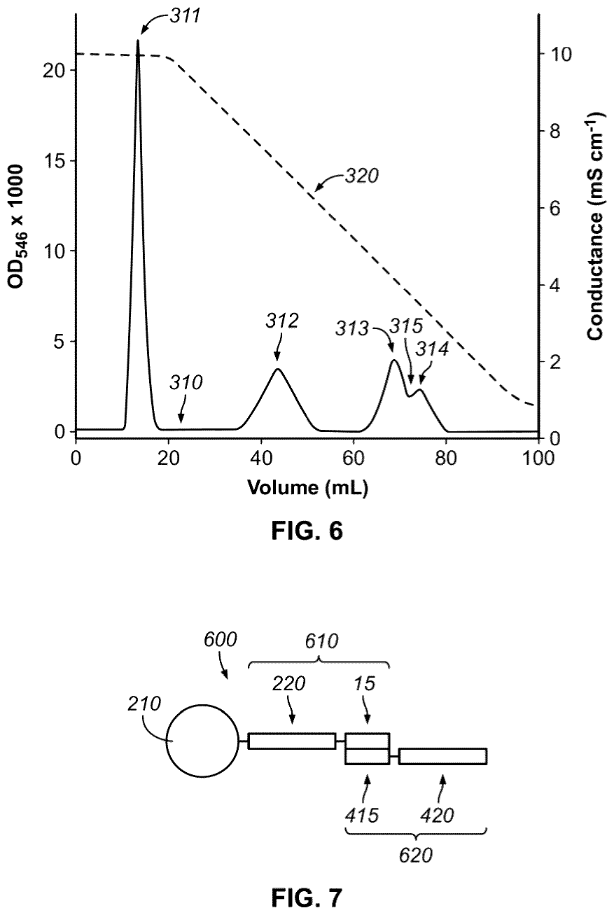

Referring to FIG. 6, after sample injection, sorting was initiated by changing the mobile phase from 100 to 0 mM [NaCl] (see curve 320) over a 75 mL linear gradient. The resulting chromatogram 310 contained four distinct peaks, 311, 312, 313, 314, corresponding to retention volumes of 13.6, 43.8, 69.3, and 74.4 mL, respectively. By contrast, 5 nm AuNPs without S1 (i.e., untagged AuNPs) subjected to the same process produced only a single peak at 13.6 mL. The observation of multiple retained peaks in the chromatogram of 5 nm S1-AuNPs suggested that separation by DNA valency was successful, and subsequent analysis of eluted peaks 311-314 by gel electrophoresis confirmed their identity as S1-AuNPs of defined valency. Crucially, S1-AuNPs eluted in order of increasing valency (untagged=peak 311, monovalent=peak 312, divalent=peak 313, tetravalent=peak 314, with trivalent appearing between peaks 313 and 314, around point 315). This result was anticipated in the design of the DNA valency sorting scheme (see, e.g., FIGS. 1A-1D) and from the experimentally measured Kd values.

Synthetic oligonucleotides were obtained from IDT with indicated purification and are listed in Table 1, below. Chemical modifications are indicated by IDT codes and the structures associated with the codes are illustrated below. Concentrations of DNA oligonucleotides were measured using the optical density at 260 nm and calculated extinction coefficients at 260 nm provided by IDT.

### Example 2

Having characterized the operation of DNA valency sorting chromatography, its scope and generality can be explored. First, it was examined whether the technique was amenable to variation in the attached DNA sequence. The selectivity of the sorting mechanism places apparent constraints on the sequence and polarity of the DNA strand attached to the nanoparticle, as it must display the keyword at its 5′-end. It was found that these constraints could be easily overcome by decoupling the keyword sequence from the nanoparticle itself. To this end, AuNPs were labeled with a 5′-bis(alkylthiol)-modified DNA strand of orthogonal sequence (S2) [SEQ ID NO. 4], which was subsequently hybridized to a temporary strand (S2′) [SEQ ID NO. 5] containing the keyword a at its 5′-end. This can be seen in FIG. 7, where the composition 600 includes nanoparticle 210 is attached to a DNA strand 610 (here, strand S2), the strand including a keyword sequence 15 and a DNA sequence 220. A second DNA strand 620 (here, strand S2′) includes a capture sequence 415 and a DNA sequence 420. The two strands form a nucleotide connection, where the keyword sequence is attached/bound to the capture sequence.

To synthesize bis(alkyl thiol)-modified S1 and S2 (S1-th2 and S2-th2), oligonucleotides S1-am2 and S2-am2 were modified with SPDP to introduce two sequential alkylthiol modifications before use (31). In a typical reaction, 100 μL oligonucleotide (100 μM in water; 10 nmol) was mixed with 40 μL 0.25 M sodium phosphate, pH 7.4, 50 μL SPDP (6.2 mg/ml in dry DMSO; 1 umol) and 10 μL water. The mixture was reacted for 1 hour at room tem-perature with gentle mixing. Excess SPDP was removed by desalting the 200 μL mixture on a NAP-5 column (GE Healthcare) following the manufacturer's instructions, and the bis(alkylthiol)-modified oligonucleotide was recovered in 700 μL 1× TE. The recovered oligonucleotide was washed twice with 1× TE and concentrated to a final volume of ˜50 μL in 1× TE using a 3000 MWCO centrifugal filter (Amicon Ultra, Millipore). The resulting S1-th2 and S2-th2 oligonucleotides were used directly without further purification.

To prepare S2′-S2-AuNPs, S2-AuNPs were prepared as for S1-AuNPs, except S2-th2 was used (instead of S1-th2) and no washing step was performed. Then, a 10-fold molar excess of S2′ was added to the S2-AuNPs and the mixture was incubated a further 2 hours prior to PEG coating. To coat with PEG, the same procedure as for S1-AuNPs was followed, except 20 mM NaCl was included in the PEG solution to maintain the S2′-S2 hybridization.

A chromatogram of 5 nm PEG-coated S2′-S2-AuNPs closely overlapped with that of S1-AuNPs, indicating that DNA valency sorting performed similarly for both single-stranded and double-stranded keyword encoding schemes. Purified S2-AuNPs could be obtained subsequent to chromatography by using toehold-mediated strand displacement to rapidly remove strand S2′. Since the sequence of S2 can be chosen freely, this simple modification ensures that a single DNA valency sorting column can be used to obtain nanoparticles tagged with a defined valency of any arbitrary DNA sequence.

### Example 3

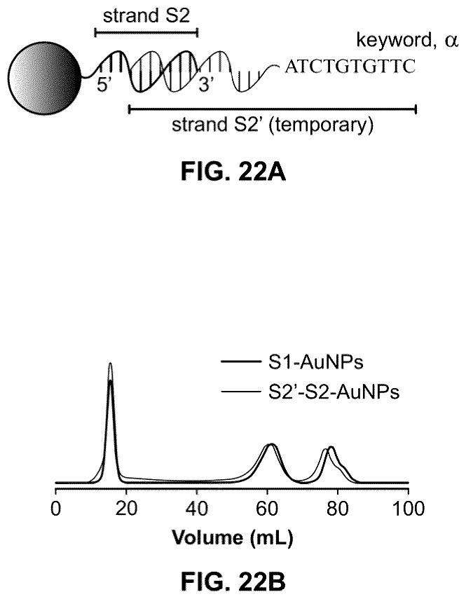

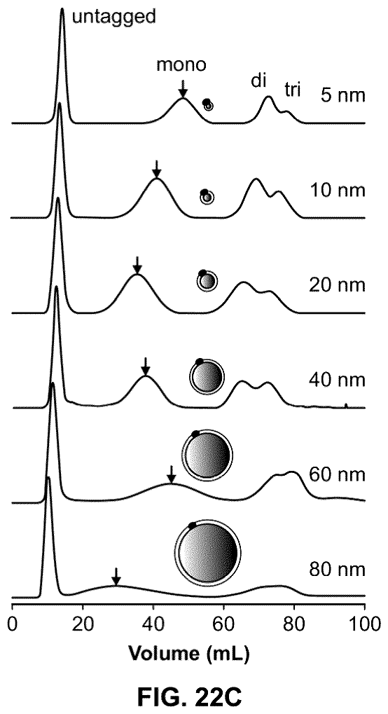

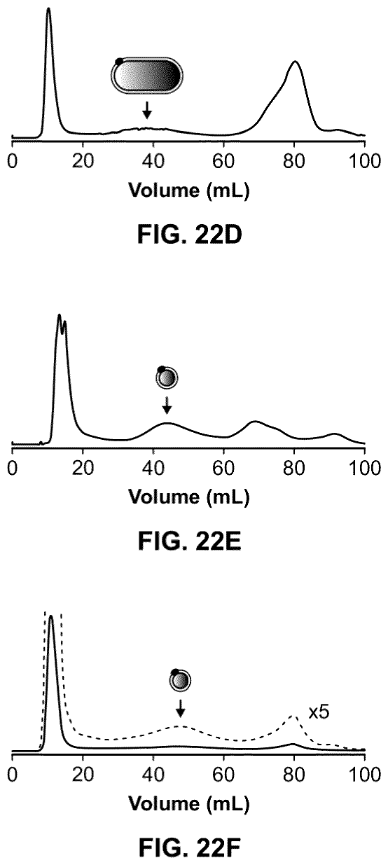

Next, DNA valency sorting was applied to different nanoparticle sizes, morphologies and materials. FIGS. 22A-22F illustrate the scope and generality of DNA valency sorting chromatography with respect to DNA sequence, nanoparticle size and shape, and nanomaterial composition, Referring to FIGS. 22A-22F, chromatograms were acquired of PEG-stabilized 5-80 nm diameter S1-AuNPs on the same column with only minor changes made to the [NaCl] in the mobile phase and flow rate used during the chromatography program. For all diameters, monovalent S1-AuNPs were resolved with baseline or near-baseline resolution. These are expected to be the largest diameter valency-defined nanoparticles that can be purified by any existing technique.

It was also found that the technique was able to resolve defined valencies of PEG-stabilized S1-tagged gold nanorods (dimensions 40×92 nm), indicating that it is effective for non-spherical, anisotropic nanoparticle shapes.

To prepare S1-gold nanorods (S1-AuNRs), first gold nanorods (AuNR) were concentrated 10-fold from the stock solution by centrifugation (2000×g, 30 min). 0.5 mL of the concentrated solution was mixed with 40 μL S1-th2 (10 μM in 1 mM BSPP), 0.5 mL of 9 mM trisodium citrate (pH 7), and 17 μL of 1% SDS. The mixture was rotated overnight. Then 200 μL 1 mg/mL COOH-PEG₂₀₀₀-SH in 1× TE was added and the mixture was further rotated for 1 hour, at which point the PEG-coated S1-AuNRs were centrifuged and concentrated to a volume of 0.15 mL in buffer A.

Additionally, DNA valency sorting was effective for nanomaterials other than gold, as it was also successful at resolving discrete S1 valencies of commercially available streptavidin-coated QDot nanocrystals or streptavidin-coated iron oxide nanocrystals tagged with biotin-labeled strand S1.

To prepare S1-QDs, 60 μL of stock streptavidin-coated QD565 (1 μM) was mixed with 60 μL S1-bio (1 μM in 1× TE, 200 mM NaCl, 0.01% SDS; 1 mol equiv) and 30 μL 1× TE, 100 mM NaCl, 0.01% SDS. The mixture was incubated for 30 min before performing DNA valency sorting.

To prepare S1-iron oxide, 150 μL stock streptavidin-coated iron oxide nanoparticles (1 mg/mL) was mixed with 23 μL S1-bio (1 μM in 1× TE, 100 mM NaCl, 0.01% SDS, 1 μM T₂₀ DNA [SEQ ID NO. 11]) and 127 μL 1× TE, 100 mM NaCl, 0.01% SDS, 1 μM T₂₀ DNA. The mixture was incubated for 30 min before directly applying to the DNA valency sorting column.

### Example 4

In some embodiments, an objective of purifying valency-defined nanoparticles is to use them as synthons in DNA-encoded nanochemical reactions. As an illustration, this example used large diameter monovalent AuNP synthons obtained from DNA valency sorting to programmatically synthesize molecularly precise artificial molecules which are challenging to synthesize with existing technology, but have potential plasmonic applications as high contrast optical labels, sensors, and single molecule optical antennas.

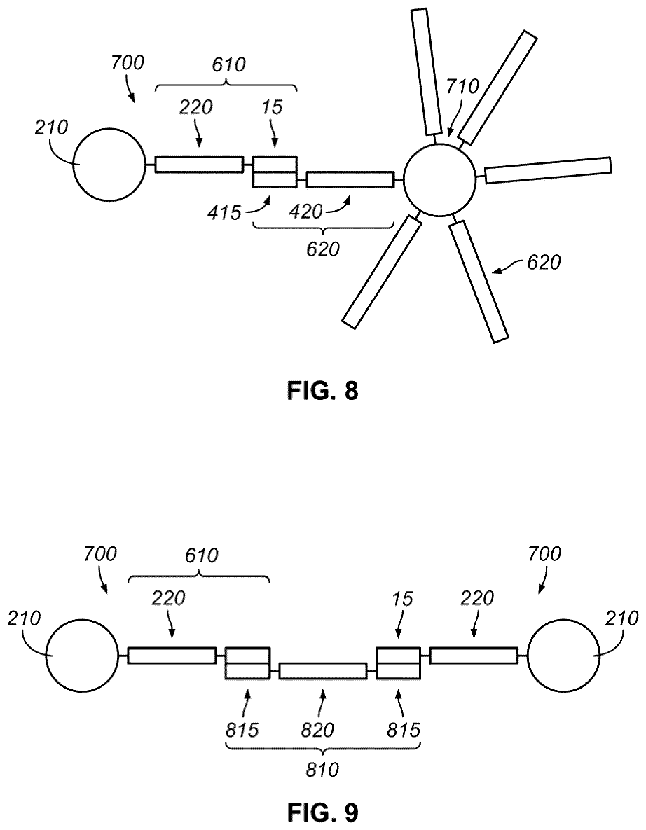

In a first approach, as illustrated in FIG. 8A, a monovalent S1-AuNP 700 (a first DNA strand 610, here S1, is shown) was interfaced with a single, smaller diameter AuNP 710 coated with multiple copies of mutually complementary sequence 620 (here, sequence S1′ [SEQ ID NO. 6]). While a smaller AuNP was used as a representative reaction partner in this illustrative example, the same synthetic scheme could be adapted to tag DNA-labeled antibodies, proteins, or biological ligands with a single AuNP for use as an optical contrast agent. The reaction proceeded by exposing monovalent 20-80 nm S1-AuNPs to a large excess of polyvalent 5, 10, or 20 nm S1′-AuNPs. After washing to remove unbound S1′-AuNPs, the reaction yield without any additional purification was assessed by counting the number of surrounding S1′-AuNPs using electron microscopy. The dimer yield was consistently ˜60% among all S1-AuNP sizes, with <15% exhibiting multivalent reactivity and the remainder ascribed to untagged AuNPs. The high yield of the expected reaction product also serves to confirm that the major species recovered from DNA valency sorting across all AuNP diameters was the intended monovalent S1-AuNPs.

To synthesize this handshake dimer, 20-80 nm S1-AuNPs were prepared, subjected to DNA valency sorting, and monovalent fractions were collected as described. Polyvalent S1′-AuNPs were prepared as follows. 5 nm S1′-AuNPs were prepared as described earlier, using 15 equivalents of S1′-th. After coating, the S1′-AuNPs were washed twice with 40 volumes 0.5× TBE using a 100 kDa MWCO centrifuge filter and concentrated. 10 and 20 nm S1′-AuNPs were prepared following a surfactant-assisted salting procedure using S′-th. After salting, the S1′-AuNPs were washed multiple times and concentrated. Monovalent 20, 40, 60 or 80 nm S1-AuNPs were mixed with 100-fold excess of 5, 10, 20, or 20 nm diameter S1′-AuNPs, respectively, in 1× TE, 100 mM NaCl, 0.01% SDS. The mixture was incubated overnight, and then excess S1′-AuNPs were removed by repeated rounds of washing by centrifugation with 1× TE, 100 mM NaCl, 0.01% SDS, 0.01% BSA. Immediately subsequent to washing, samples were visualized by TEM.

In a second approach, a dimer was formed by docking two identical monovalent AuNPs 700 onto a preformed dsDNA template 810. The dsDNA template includes a two capture sequences 815 separated by a DNA sequence 820. The keyword sequence on each nanoparticle bind to one of the capture sequences on the template.

Since the localized surface plasmon resonance (LSPR) is sensitive to the interparticle distance, similar dimeric structures can act as plasmonic sensors that optically reports on DNA conformation, enzymatic activity, or ligand binding. The reaction was performed by mixing monovalent S1-AuNPs with a template dsDNA (T11). T11 is comprised of two sequences—T11a [SEQ ID NO. 7] and T11a′ [SEQ ID NO. 8]—that include identical capture sequences for the keyword sequences attached to the nanoparticles (here, 30-nt sequences that are complementary to the keyword sequence in the S1 attached to the AuNPs), and a 21-nt sequence that are reverse complements of each other, allowing the two overall sequences (here, T11a and T11a′) to bind together while keeping the two capture sequences free to bind to the keyword sequences.

To synthesize the template-directeted S1-S1 homodimers, 20-80 nm S1-AuNPs were prepared, subjected to DNA valency sorting, and monovalent fractions were collected. Optimal reaction conditions were determined in a series of small-scale reactions. The general procedure was as follows. S1-AuNPs were diluted to a desired concentration with reaction buffer (1× TE, 1 μM T₂₀, 150-200 mM NaCl). Typical concentrations were 10, 1, 0.2, and 0.05 nM for 20, 40, 60 and 80 nm. S1-AuNPs were mixed with T11 in T11:S1-AuNP molar ratios of 0.25, 0.40, 0.65, 1.1 and 1.9:1. Typical reaction volumes were 4-5 μL. To prepare the double-stranded DNA template T11, oligonucleotides T11a and T11a′ (each 20 μM) were mixed in equal proportions in 1× TE buffer containing 100 mM NaCl and annealed by heating at 95°° C. for 2 minutes in an aluminum block followed by gradual cooling to room temperature over ˜2 hours. After incubating 12-48 hours, the reactions were analyzed to determine dimer yield. For 20 nm AuNPs, reactions were assayed by agarose gel electrophoresis and visual inspection was sufficient to identify the condition with maximum dimer yield. For 40-80 nm AuNPs, reactions were analyzed by UV-Vis spectroscopy. The condition which produced maximum dimer yield was assumed to correspond to that with the largest shift in the wavelength of the surface plasmon resonance maximum (λmax). λmax was determined by fitting the peak of the spectrum to a gaussian in MATLAB. A plot of λmax versus [template] has triangular form, and the condition at the peak was identified as optimal.

Once the optimal condition was identified, it was scaled-up by simply increasing the total volume of reaction (10-50 μL). This scaled up reaction was incubated as before and used for further characterization. As expected, for sufficiently large AuNPs (>20 nm), the homodimer product exhibited plasmonic coupling that could be detected spectroscopically by a shift in the LSPR maximum. In all cases, the expected S1-S1 homodimer was the only significant product and was obtained in ≥50% yield across multiple replicate experiments. These results indicate that programmable nanostructure synthesis is possible regardless nanoparticle size, as long as the required purified reagents are available.

As a final approach, a distinct dimeric structure was formed by “clamping” monovalent S1- and S2-AuNPs together with an additional DNA strand (T12, [SEQ ID NO. 9]) which was modified with a single Cy®3 mono NHS Ester molecule. This can be seen in FIG. 9, where a first nanoparticle 700 (here, S1-AuNP) and a second nanoparticle 701 (here, S2-AuNP) are coupled to a linking DNA strand 910 (here, T11), which includes capture sequences 915, 916 for binding to the keyword sequences 15, 16 of S1 and S2, respectively.

The single-stranded “clamping” template T12-Cy3 was prepared by reacting oligonucleotide T12-am with Cy®3 mono NHS ester. In a typical reaction, 30 μL T12-am (500 μM in water; 15 nmol) was mixed with 20 μL Cy®3 mono NHS ester (10 mg/mL in dry DMSO; 260 nmol) and 20 μL 0.25 M sodium borate, pH 8.5, and gently mixed for two hours at room temperature. To remove excess Cy®3 mono NHS ester, the mixture was diluted to 100 μL with 1× TE and desalted on a NAP-5 column (GE Healthcare), recovering the modified oligonucleotide in 500 μL 1× TE. The recovered oligonucleotide was further purified and concentrated by ethanol precipitation. The degree of Cy®3 mono NHS ester labelling was quantified by comparing the concentrations of DNA and Cy®3 mono NHS ester, measured by optical density at 260 nm and 550 nm, respectively. The typical labelling percentage was 95%.

To synthesize the clamping S1-S2 heterodimer, 80 nm S2′-S2-AuNPs were prepared, subjected to DNA valency sorting, and monovalent fractions were collected as described. To obtain S2-AuNPs, invading strand S2″ was added in 100-fold molar excess to S2′-S2-AuNPs. After incubating for 12-18 hours, the sample was washed to remove unbound DNA strands. 80 nm S1-AuNPs were prepared as above. To enable specific immobilization onto coverslips for single molecule microscopy, 0.4% biotin-PEG₂₀₀₀-lipoamide was included during PEG coating of the S1-AuNPs prior to DNA valency sorting. The ssDNA complex T12-Cy®3 was used as a clamp and reaction screening to identify optimal conditions was performed as for the S1-S1 homodimer. S1-and S2-AuNPs were mixed in equal concentrations and T12-Cy®3 was added in approximate T12-Cy®3:S1-AuNP:S2-AuNP molar ratios of 0.5, 0.8, 1.3, 2.2, and 3.7:1:1. The optimal condition was identified and scaled up as described above.

Plasmonic coupling in the dimer is expected to generate an enhanced local electromagnetic field at the interparticle gap, causing it to act as an optical antenna which amplifies the emission of the associated Cy®3 mono NHS ester molecule. The results of a representative reaction using 80 nm AuNPs are shown without further purification. Similar to the S1-S1 homodimer reaction, gel densitometry revealed that the S1-S2 heterodimer was obtained in ˜52% yield, and plasmonic coupling was again indicated by a red-shift in the LSPR spectrum.

To confirm that the 80 nm dimer acted as an optical antenna, the reaction mixture was characterized using consecutive single particle TIRF and darkfield scattering microscopies.

To perform single particle TIRF and darkfield scattering microscopy of 80 nm dimer optical antennas, first coverslips were passivated as follows to immobilize biotin-PEG-coated particles through strepatividin-biotin. Coverslips (24×40 mm, No. 1, Fisherbrand) were cleaned by heating in IM NaOH at 55° C. for 1 hour. After cleaning they were washed extensively with water and ace-tone, incubated for 1 hour in 3% v/v 3-aminopropyltriethoxy silane in acetone, washed extensively with acetone and water, then dried under nitrogen. A solution containing 187.5 mg/mL mPEG₅₀₀₀-SVA and 12.5 mg/mL biotin-PEG₅₀₀₀-SVA was prepared in 0.1 M sodium borate, pH 8.5. 90 to 100 μL of the PEG solution was applied to one coverslip and another was placed on top to form a sandwich. The coverslip sandwiches were incubated overnight in a humidified chamber. Finally, the biotin-PEG-coated coverslips were washed extensively with water and stored upright until use.

Next, a home-built flow cell was constructed on the coverslips to facilitate sample application. An adhesive silicon spacer with a 13 mm diameter circular opening (Coverwell, Grace Bio) was cut in half and both halves were adhered onto a biotin-PEG-coated coverslip, leaving a gap of 1-3 mm between them. A dust free coverslip (18×18 mm, No. 1, Fisher brand) was placed on top. Liquid was flowed through the chamber by pipetting a drop on one end and wicking at the other end with filter paper. To immobilize particles, the flow cell was washed twice with 50 μl buffer (1× TE, 200 mM NaCl). 20 μL streptavidin (Prospec bio; 200 nM) in the same buffer was flowed through the cell two times, and the cell was incubated for 20 minutes in a humidified chamber, then washed ten times with 50 μL buffer. 10 μL of the 80 nm dimer reaction mixture (typically diluted to 10-30 pM AuNPs in buffer) was flowed through the cell twice, and the cell was incubated for 20 minutes in a humidified chamber in the dark, followed by washing two to three times with 20 μL buffer. Finally, the chamber was sealed permanently with nail polish to prevent evaporation.

The single particle microscope described by Li and Yang was used to acquire TIRF and darkfield (DF) images. TIRF and DF images of a field of view were acquired consecutively by using a 100× oil-immersion objective with an adjustable NA collar. To acquire TIRF images, the NA was opened to 1.4, the sample was excited with a 532 nm laser, and emission was collected through a 560 lp filter onto an water-cooled EMCCD (Andor, iXon). Images were collected at a rate of 1 frame per second (200 ms exposure time) for 120 s total. Subsequently, the objective NA was stopped down to 0.7, the laser was blocked with a shutter, and the sample was illuminated with a halogen lamp through an oil-immersion darkfield condenser. A single dark-field image of the field of view as acquired (200 ms exposure). The sample was manually moved to reveal another field of view and the process was repeated. Typically, 5-10 fields of view were acquired.

The single particle images were analyzed in Matlab to identify dimer structures associated with Cy®3 mono NHS ester and quantify the emission intensity of the associated Cy®3 mono NHS ester molecule.

First, the DF image was registered to the corresponding TIRF image (at time=0 seconds) by intensity-based image registration in Matlab. Only rigid geometric transformation as permitted (translation and rotation). Next, the location of local maxima, corresponding to individual particles, were identified in the DF image using the function FastPeakFind (Adi Natan, 2013). Maxima were only identified if they exceeded an empirically determined threshold, specified as μ+0.8 σ, where μ is the average pixel intensity and σ was the standard deviation of pixel intensity in the image. The DF and TIRF intensity at each particle location and time point was determined by averaging a 3×3 pixel region surrounding the identified maxima. The resulting values were divided by the exposure time (200 ms). The location of the local maxima were assumed to correspond to the particle locations. For the dsDNA-Cy3 reference, DF images were not registered and the local maxima identification procedure was performed using only the initial TIRF image (time=0).

Next, the log(DF) and log(TIRF) intensities were used to group observed particles into populations by k-means clustering using function kmeans, where the number of clusters was chosen based on the Calinski-Harabasz criterion using function evalclusters. Once clustered, populations were identified as monomer, dimer, monomer-Cy3, dimer-Cy3, or debris (unclassified) based on their relative location in the plot and by comparison to controls. (No clustering was performed on the dsDNA-Cy3 reference as all observed spots were assumed to correspond to a single population.)

After clustering, the TIRF intensity versus time for each Cy3-containing species (monomer-Cy3, dimer-Cy3, dsDNA-Cy3) was manually inspected. Data were excluded from further analysis if they met any of the following conditions: (i) no bleach step, (ii) more than one bleach step, (iii) multiple on/off intensity changes. Typically, 25% of data were excluded. Each retained TIRF trajectory was analyzed to obtain the background-subtracted TIRF intensity by subtracting the average TIRF intensity after bleaching from the average value before bleaching.

Referring to FIG. 11, dimers enhanced the fluorescence of Cy®3 mono NHS ester 1000 by on average 4-fold compared to a control dsDNA structure 1001. To prepare the dsDNA-Cy3 complex used as a reference in single molecule analysis of 80 nm dimer optical antennas, oligonucleotides S1-bio, S2, and T12-Cy3 were mixed in a 1.28:1.28:1 ratio in 1× TE, 150 mM NaCl and annealed as for template T11.

Additionally, since a large amount of monovalent S1- and S2-AuNPs could be isolated with DNA valency sorting, it was possible to synthesize sufficient optical antennas for characterization by ensemble fluorescence spectroscopy.

To perform ensemble fluorescence spectroscopy of 80 nm dimer optical antennas, the 80 nm S1-S2 heterodimer reaction (20 μL) was diluted with 125 μL buffer (0.7× TE, 200 mM NaCl, 0.1% BSA) and centrifuged at 4° C. After centrifugation, the supernatant was removed to obtain a final volume of 20 μL in the same buffer. The washed sample (10-15 μL) was diluted with 200 μL buffer (0.7× TE, 200 mM NaCl) and transferred to a 3×3 mm quartz cuvette (Starna). Typical OD₅₂₅ was 0.05 to 0.1. Fluorescence emission spectra were acquired by exciting at a wavelength of 525 nm (5 nm slit width) and collecting emission at 90° through a 540 LP filter (Omega optics). The top of the cell was covered with foil and the sample chamber was thermostatted at 20° C. with a circulating water bath. For each measured sample, three consecutive fluorescence spectra were acquired. UV-Vis extinction spectra were acquired in the same 3×3 mm cuvette before fluorescence measurements.

The emission enhancement of Cy®3 was assessed using an in-situ reference procedure. First, UV-Vis and fluorescence spectra were acquired for the dimer reaction mixture. Then the T12-Cy3 was decoupled from the nanoparticles by adding invading strand T12′ [SEQ ID NO. 10] (1 μL of 10 μM) and incubating for 30 minutes in the dark. After incubation, UV-Vis and fluorescence spectra were acquired for this reference solution as before. Successful disassembly of the dimers was confirmed by the shift in the λₘₐₓ of the UV-Vis spectrum.

To analyze the data, fluorescence spectra were first corrected for dark counts, wavelength-dependent detector sensitivity, variations in excitation intensity, and the inner filter effect. The three consecutive spectra obtained for each solution were averaged and the standard deviation at each wavelength was calculated and used as the measurement uncertainty. The background of each observed spectrum was estimated using a known spectral unmixing procedure. Briefly, the observed spectrum was fit to a linear combination of weighted spectral components, corresponding to the unspecified background (measured from a solution of similar AuNPs without Cy®3), the water raman signal (observed at ˜640 nm; measured from a water blank), Cy®3 emission, and a constant. Then the background was calculated as the sum of the unspecified background, water raman, and constant term (with weights obtained by non-linear least squares regression) and subtracted from the observed spectrum. After background subtraction, the reference spectra were additionally corrected for the effect of dilution by multiplying by the experimentally measured dilution factor. Then both initial and reference spectra were normalized to the maximum intensity of the reference.

Bulk fluorescence spectra also indicated a comparable magnitude of enhancement. It was noted that similar dimeric optical antennas have been reported in the literature, but their synthesis often proceeds in low yield or requires surface-immobilized DNA origami scaffolds. By contrast, this example illustrates that with monovalent AuNP synthons obtained by DNA valency sorting, molecularly-defined optical antennas can be directly obtained in large amounts and high yield in a one-step reaction. The purified synthons that make these pieces of nanochemistry possible were generated in large quantities by the DNA valency sorting chromatography introduced here. It is general, applicable to any DNA sequence and a range of nanoparticle sizes, shapes, and material compositions, including the formerly inaccessible large metallic nanospheres and nanorods, as well as quantum dots and iron oxide nanocrystals. With it, it can be show that the digital information encoded in the number and sequence of DNA molecules on a nanoparticle not only facilitates programmatic synthesis of artificial molecules, but programmable purification of valency-defined nanoparticles as well. With a large amount of purified synthons, DNA-encoded nanochemical reactions can generate artificial molecules with sufficient crude yield for subsequent reactions in multi-step synthesis or in-depth study by ensemble spectroscopy, thereby suggesting much expanded possibilities for the synthesis and interrogation of designer nanostructures.

Samples for the above experiments were prepared for injection by dispersing DNA-tagged nanoparticles into buffer A. For AuNPs, typical OD₅₂₀ of the input samples were 1-20 AU. The general chromatography method was as follows. The column was equilibrated with buffer A, the sample (0.1-0.4 mL) was injected, and the mobile phase was changed from 0 to 100% buffer B over 75 mL, followed by a 35 mL wash with 100% buffer B. Buffer B was composed of 1× TE, 0.01% SDS. Buffer A was composed of 1× TE, 0.01% SDS and NaCl. The amount of NaCl in buffer A and the flow rate varied depending on the nanoparticles being analyzed, as follows. 5-20 nm AuNPs: 100 mM NaCl, 0.4 mL/min; 40 nm AuNPs: 150 mM NaCl, 0.1 mL/min; 60 nm AuNPs: 200 mM NaCl, 0.1 mL/min; 80 nm AuNPs: 280 mM NaCl, 0.1 mL/min; Au nanorods: 280 mM NaCl, 0.1 mL/min; streptavidin QDs: 100 mM NaCl, 0.4 mL/min; streptavidin iron oxide nanoparticles: 250 mM NaCl, 0.1 mL/min. AuNPs and nanorods were detected by monitoring OD at 546 nm. QDs were detected at 254 nm. Iron oxide nanoparticles were detected at 436 nm. For preparative experiments, automated fractions were collected in 2 to 4 mL increments through the chromatography process. Fractions corresponding to the desired peak(s) were pooled and concentrated. For 5-10 nm AuNPs, AuNPs were concentrated using a 30kDa MWCO centrifugal filter (Amicon Ultra, Millipore) and washed at least once with buffer (1× TE, 0.01% SDS, 50 mM NaCl). For 20-80 nm AuNPs, 1/10v olume 1% BSA (0.2 μm sterile filtered) was added to the pooled fractions and they were concentrated by centrifugation and washed once with buffer (1× TE, 0.01% SDS, 100-200 mM NaCl). To process and analyze chromatograms, baselines were estimated with Prime View software and subtracted.

TABLE 1

| | Purifi- | |
|---|---|---|
| Name | cation | Sequence (5′ to 3′) |
| α-T₁₅ | None | TAGACACAAG TTTTTTTTTT TTTTT |
| α-T₁₅- | None | TAGACACAAG TTTTTTTTTT TTTTT/ |
| am | | 3AmMO/ |
| S1-am2 | HPLC | CTTGTGTCTA TCCCCATCTA CCTTTCTCTT |
| | | TACATCATCC ACCTTTTTTT TTT-/ |
| | | iUniAmM//3AmMO/ |
| S1-bio | None | CTTGTGTCTA TCCCCATCTA CCTTTCTCTT |
| | | TACATCATCC ACCTTTTTTT TTT-/3Bio |
| S1′-sh | None | AAGAGAAAGG TAGATGGGGA TAGACACAAG |
| | | TTTTTTTTTT-/3ThioMC3-D/ |
| S2 | PAGE | TTTTTTTTTT CCACCTACTA CATCTTAATC |
| | | CTAGCTTAAT ACTTTTCATT CTC |
| S2-am2 | HPLC | /5AmMC6//iUniAmM/-TTTTTTTTTT |
| | | CCACCTACTA CATCTTAATC CTAGCTTAAT |
| | | ACTTTTCATT CTC |
| S2′ | PAGE | CTTGTGTCTA TTTTTTTTTG AGAATGAAAA |
| | | GTATTAAGCT AGGATTAAG |
| S2″ | PAGE | CTTAATCCTA GCTTAATACT TTTCATTCTC |
| | | AAAAAAAAA |
| T11a | PAGE | AAGAGAAAGG TAGATGGGGA TAGACACAAG |
| | | TATATTCTAA AAGTTCCAAC C |
| T11a′ | PAGE | AAGAGAAAGG TAGATGGGGA TAGACACAAG |
| | | GGTTGGAACT TTTAGAATAT A |
| T12-am | HPLC | TAGGTTCCAA GAGAAAGGTA GATGGGGATA |
| | | GACACAAGGA GAA/iAmMC6T/GAAAAGTATT |
| | | AAGCTAGGAT TAAG |
| T12′ | None | CTTAATCCTA GCTTAATACT TTTCATTCTC |
| | | CTTGTGTCTA TCCCCATCTA CCTTTCTCTT |
| | | GGAACCTA |
| T₂₀ | None | TTTTTTTTTT |

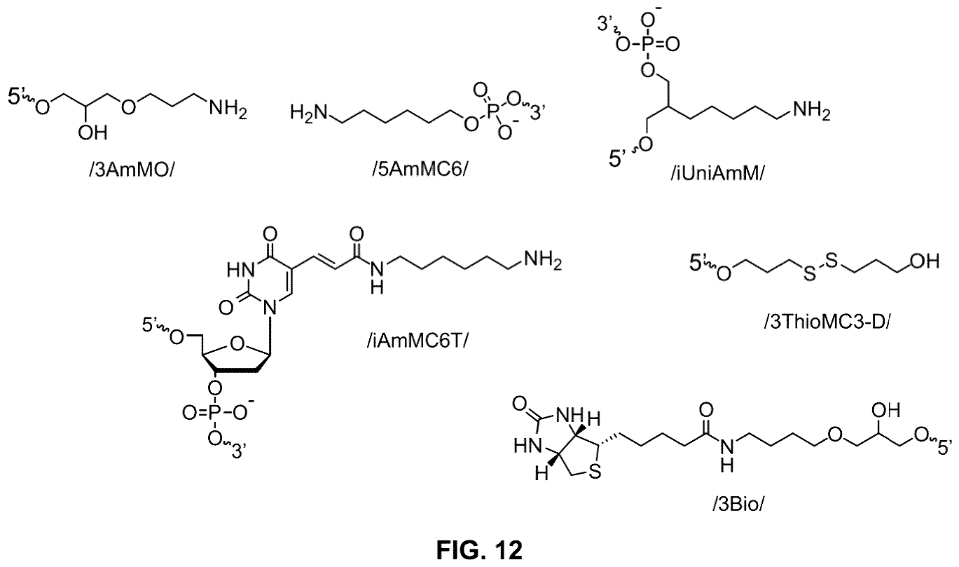

In the table above, several chemical structures are indicated. The structures indicated are shown in FIG. 12.

### Example 5

The nanoparticle-bioconjugate is a hybrid system which combines the biological recognition and chemical reactivity of the attached molecules with the unique, size-dependent optical, electronic, and physical properties of the nanomaterial. Although nanoparticles are often uniformly coated with a biomolecule of interest, greater chemical and biological control can be achieved by using nanoparticles bearing a distinct number, or valency, of molecules. These discrete nanoparticle-bioconjugates are useful reagents for self-assembling molecule-like or extended nanocomposites in solution and for precisely labeling, tracking, or manipulating single proteins in biological system. They also have important implications for nanomedicine, where controlling the number of molecules on the nanoparticle surface enables one to tailor the magnitude of cell binding, uptake, and receptor activation. Unfortunately, routine preparation of discrete nanoparticle-bioconjugates remains challenging, because the lack of regioselectivity leads most conjugation reactions with nanoparticles to generate a mixture of products. A few specialized synthetic approaches can favor the production of a single desired product, but they are highly specific for particular nanoparticles and biomolecules and often require purification at some stage of synthesis. As a result, high-resolution methods which can separate discrete nanoparticle-bioconjugates are necessary for either analytical or preparative downstream applications.

While a number of conventional purification techniques have been applied to nanomaterials, few are effective at separating discrete nanoparticle-bioconjugates. One widely used technique is gel electrophoresis, in which solutes are sieved through a porous gel matrix under an electric field and resolved by differences in size and/or charge. Gel electrophoresis is largely indiscriminate to the identity of the attached molecule, enabling it to separate nanoparticles bearing a discrete number of, for example, DNA oligonucleotides, proteins, and polymers. Despite its generality, resolution in gel electrophoresis is often low and material losses occur when extracting species from the gel. Liquid chromatography has emerged as a promising alternative, owing to its potential for achieving rapid, scalable, and high-resolution separations of biomolecules. A variety of modalities have been applied to the separation of nanoparticle-molecule conjugates with varying success, including size-exclusion, HPLC, anion-exchange, and affinity. Yet, applications of liquid chromatography techniques to the separation of nanomaterials are still in their infancy, and most of the aforementioned chromatography modalities have thus far only been demonstrated for a narrow range of nanoparticle-bioconjugates.

Nanoparticle-bioconjugates are distinct from the isolated molecule in important ways which can adversely affect the outcome of separation, but which are not well understood. First, since nanoparticles (1-100 nm) span a much larger length scale than biomolecules (1-10 nm) the size of the nanoparticle-bioconjugate is often dominated by the nanoparticle rather than the attached biomolecule(s). This leads to a predictable deterioration in resolution as nanoparticle dimensions increase for size-dependent separation schemes, such as gel electrophoresis. Yet, protein studies indicate that increasing solute size may also impact outcome in affinity and anion-exchange chromatography, which do not explicitly depend on size. Second, the shell of coordinated passivating ligands, required to stabilize colloidal nanocrystals in aqueous solution, dominates the chemical and electrostatic properties of the nanoparticle. This can interfere with selective purification techniques, as observed in anion-exchange chromatography of DNA-labeled nanoparticles, where high molecular weight, negatively-charged ligands severely compromised resolution. Finally, immobilization of biomolecules onto the nanoparticle surface, often within the layer of passivating ligands, can alter their structure and function, possibly perturbing their interaction with the separation medium relative to the free biomolecule.

To extend liquid chromatographic purification of discrete nanoparticle-bioconjugates to more nanomaterials, morphologies, and biomolecules, as well as to improve the resolution of existing techniques, it is necessary to understand in more detail how the unique characteristics of the nanoparticle affect the outcome of a separation. In this article, we address this question by systematically studying the impact of two important variables, nanoparticle size and ligand coating, on retention in DNA valency sorting chromatography, an affinity chromatography technique recently introduced by our group. We find that nanoparticle diameter and surface coating both can have significant impacts on separation outcome. We experimentally rationalize these effects in terms of the physical attributes of the nanoparticle solute as well as fundamental chromatographic or thermodynamic parameters. Finally, we demonstrate how this newly developed understanding can inform practical approaches to overcome the negative impacts of diameter and surface coating and rationally improve the resolution of previously difficult-to-separate discrete nanoparticle-bioconjugates.

Strand S1 was anchored onto the gold surface through two sequential thiol modifications at its 3′-end and contained the a sequence at its 5′-end to permit interaction with the capture resin. The S1-AuNPs were additionally coated with a ˜350 Da methoxy-PEG-thiol ligand (mPEG₆-SH, PEG 1 in Table 2) to resist aggregation and non-specific adsorption during chromatography. The sample was applied to a DNA valency sorting column and chromatographed by decreasing [NaCl] from 100 mM to 0 mM over 75 mL at a flow rate of 0.4 mL/min. Distinct peaks were observed during the gradient, which, as discussed previously, are identified these peaks as AuNPs with well-defined S1 valency, establishing that retention of a nanoparticle solute in DNA valency sorting is governed by its α valency and affinity for the capture resin.

TABLE 2

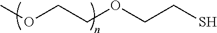

1: n = 6, mPEG₆-SH
3: n ≈ 16, mPEG₈₀₀-SH
6: n ≈ 45, mPEG₂₀₀₀-SH
7: n ≈ 114, MPEG₅₀₀₀-SH

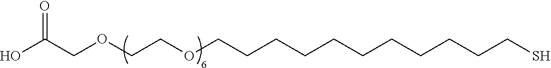

2: COOH-PEG₆-C₁₁-SH

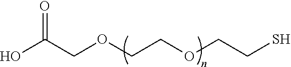

4: n ≈ 22, COOH-PEG₁₀₀₀-SH
5: n ≈ 45, COOH-PEG₂₀₀₀-SH

This investigation into the impact of nanoparticle characteristics in DNA valency sorting chromatography centers on this model system of PEG-stabilized S1-tagged AuNPs. The modularity of this system enables the diameter of the nanoparticle and the identity of the PEG coating to be varied independently. To investigate the effect of nanoparticle diameter on separation outcome, a series of S1-AuNPs with nominal AuNP diameters of 10 to 60 nm were prepared. They were stabilized with the same carboxymethyl hexa(ethylene glycol) undecane thiol ligand (COOH-PEG₆-C₁₁-SH, PEG 2 in Table 2). This ligand was chosen as it was the smallest PEG which could effectively stabilize this range of AuNPs sizes in 100 mM NaCl application buffer required for DNA valency sorting (data not shown). To investigate the effect of PEG coating on separation outcome, a series of S1-AuNPs with nominal AuNP diameter of 20 nm were prepared and stabilized with seven different alkyl-thiol PEG ligands (Table 2). The PEG ligands spanned a molecular weight range of 350 to 5000 Da, as well as both neutral (methoxy) and negatively charged (carboxy) terminal groups. The hydrodynamic diameters (Dₕ) of the identical AuNPs coated with only the PEG ligand were measured by dynamic light scattering (DLS). The obtained values are summarized in Table 3 and were used as an estimate of solute size in the foregoing analysis.

TABLE 3
Diameters of PEG-stabilized AuNPs used in the study.

| PEG Ligand | | Diameter (nm) | | |
|---|---|---|---|---|
| Identity | MW (Da)ª | Nominal | DLS (Dₕ)^{b} | TEM^{c} |
| 2 | 526 | 10 | 18.9 ± 0.1 | 10.5 ± 1.0 |
| 2 | | 20 | 24.9 ± 0.5 | 17.6 ± 1.4 |
| 2 | | 40 | 46.9 ± 1.0 | 40.1 |
| 2 | | 60 | 66.0 ± 1.3 | 59.0 |
| 1 | 356 | 20 | 21.6 ± 0.2 | 17.6 ± 1.4 |
| 3 | 791 | | 24.6 ± 0.3 | |
| 4 | 1200 | | 26.4 ± 0.3 | |
| 5 | 2000 | | 30.9 ± 0.2 | |
| 6 | 1900 | | 30.4 ± 0.4 | |
| 7 | 4800 | | 41.0 ± 0.6 | |

^{a}Molecular weight, reported by the supplier.
^{b}Hydrodynamic diameter from dynamic light scattering, reported as mean ± standard deviation of at least five measurements.
^{c}Diameter obtained from transmission electron microscopy, reported by the supplier.

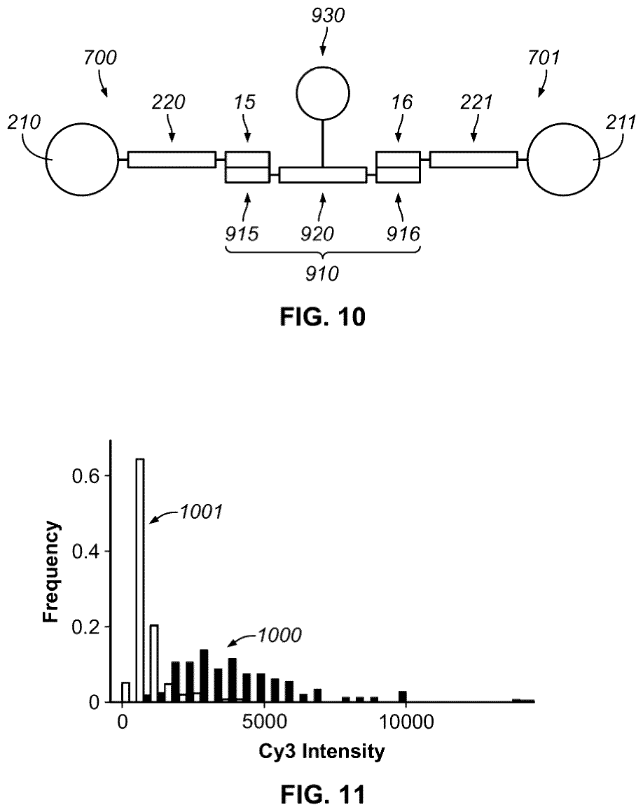

Effect of Diameter. The effect of nanoparticle diameter on separation outcome was investigated in a first set of experiments. The 10-60 nm S1-AuNP samples stabilized with PEG 2 were subjected to DNA valency sorting on the same column using the same method as for the 5 nm S1-AuNPs. As shown in FIGS. 13, 10 and 20 nm diameter S1-AuNPs produced similar chromatograms containing multiple distinct peaks, indicative of the successful separation of the input mixture into individual S1-AuNP valencies. The observed peaks were identified as unlabeled AuNPs, and mono-, di-, and tri-valent S1-AuNPs based on their relative retention values, as well as their correspondence with peaks in the chromatogram of 5 nm S1-AuNPs. For 40 and 60 nm diameter S1-AuNPs, retained peaks were still observed, but they exhibited considerable overlap, and only unlabeled and monovalent species could be distinguished with confidence. This series of chromatograms demonstrated that, for a fixed chromatography method, the quality of the separation was dependent on the diameter of the AuNPs, with the overall resolution deteriorating significantly for diameters >20 nm.

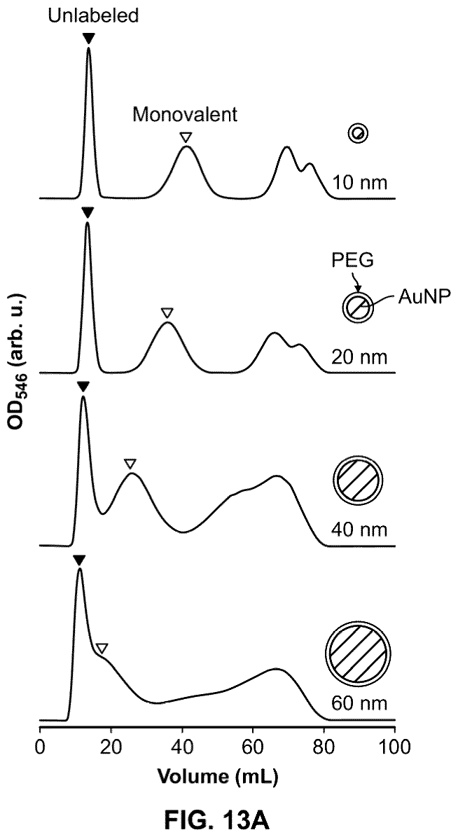

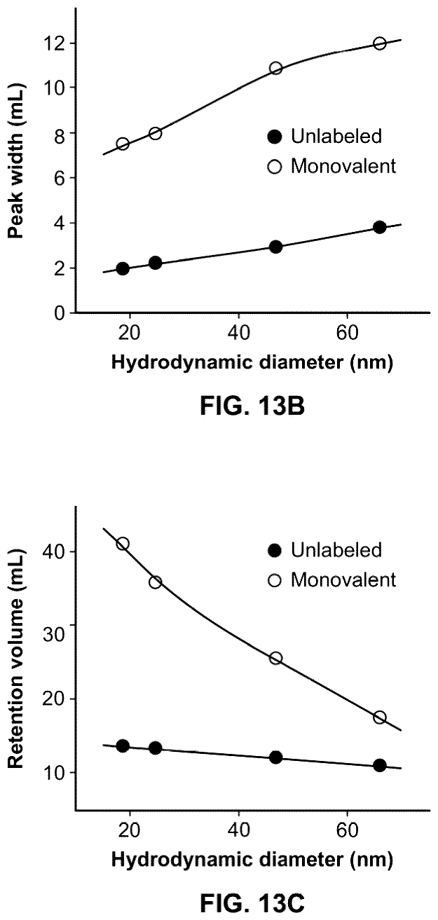

To gain insight into the origin of the observed size-dependence, the retention and peak width of the indicated unlabeled and monovalent peaks were analyzed as a function of hydrodynamic diameter, Dₕ. Analysis of the chromatograms revealed that peak width increased with increasing Dₕ (FIG. 13B). This correlation is expected, because two of the major sources of dispersion in a chromatography system—longitudinal diffusion and resistance to mass transfer—depend on the solute's diffusion coefficient, which is an explicit function of solute size. The relatively slow association and dissociation rates of the selective affinity interaction are also an important contribution to dispersion. Experimental studies have reported that the hybridization kinetics of DNA oligonucleotides are altered when they are immobilized onto planar surfaces, as well as onto micro- or nanoparticles, suggesting that DNA interactions are dependent on their local physical environment. Yet, the precise origin of these effects and the role of variables such as the particle size and immobilization density are unresolved. Although we did not characterize the kinetics of the underlying α-α′ affinity interaction at this time, and without being held to any particular theory, it is speculated that they are likely a contributing factor to the size-dependence of resolution observed here.

In addition to changes in peak width, retention also varied for both unlabeled and monovalent species as AuNP diameter increased (FIG. 13C). For unlabeled AuNPs, the observed decrease in retention with increasing size is characteristic of size-exclusion. This result is expected, because the Dₕ of the AuNPs studied here fall within the reported selectivity range of Sephacryl S-1000 (20-200 nm), which constitutes the base support. Perhaps the most significant size-dependent effect was the dramatic decrease in retention of monovalent S1-AuNPs with increasing diameter. This behavior could also be attributed to size-exclusion, which can have an outsized influence on retention for interacting solutes. For example, studies of conventional affinity and anion-exchange chromatography of proteins have reported that binding capacity, retention, and/or separation efficiency diminished as protein molecular weight approached the exclusion limit of the base resin. This phenomenon is understood to occur because a majority of the surface area, and therefore a majority of the ligand, resides in the resin pores. When larger solutes are excluded from much of the pore volume, they experience an effectively lower local concentration of the ligand, which modulates the equilibrium adsorption behavior and, consequently, binding capacity and retention.

To test whether size-exclusion could explain the magnified decrease in retention of larger S1-AuNPs, the retention of a fixed size (20 nm) of purified monovalent S1-AuNPs on small-scale DNA valency sorting columns containing resins with different exclusion properties was analyzed. Three columns were prepared with α′-modified Sephacryl® S-200, S-300, and S-400, which have estimated maximum pore dimensions of 14, 26 and 70 nm, respectively. The column volume (˜1.5 mL), the concentration of α′ (10 nmol per g wet resin), and the average particle size (50 μm) were held constant, leaving the pore dimension as the only variable. Since these pore dimensions comprise a range which spans both below (S-200) and above (S-400) the hydrodynamic diameter of the 20 nm AuNPs (Table 3), this solute will experience distinct size-exclusion behavior on each column. If size-exclusion indeed modulates the retention of S1-AuNPs as described above, then the 20 nm monovalent S1-AuNPs should exhibit distinct retention values on the three columns.

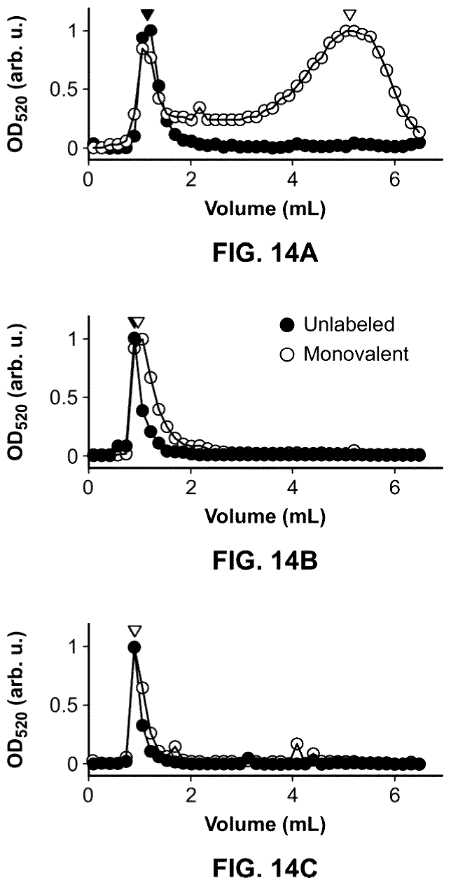

Chromatograms were acquired on miniature (1.5 mL) columns packed with α′-modified resins: Sephacryl® S-400 (FIG. 14A), Sepahcryl® S-300 (FIG. 14B), and Sepahcryl® S-200 (FIG. 14C). Samples (20-35 μL) were applied in buffer containing 100 mM NaCl and, after washing with 1.6 mL application buffer, were eluted with a manual gradient in which [NaCl] was incrementally reduced to 0 mM over 3.2 mL. Equal fractions (0.16 mL) were collected throughout and absorbance was measured to construct the chromatograms shown.

FIGS. 14A-14C show chromatograms of 20 nm monovalent S1-AuNPs and corresponding unlabeled AuNPs obtained on each column. As for the typical DNA valency sorting procedure, chromatography was performed by incrementally reducing mobile phase [NaCl] from 100 mM to 0 mM. Qualitatively, it is evident that the behavior of the 20 nm monovalent S1-AuNPs varied significantly across the different columns. For example, S1-AuNPs were highly retained on the S-400 column (FIG. 14A), where the typical pore size was much larger than the AuNPs (it is noted that a small amount of unlabeled AuNPs are present in the monovalent S1-AuNP sample and are visible in the chromatogram). By contrast, monovalent S1-AuNPs were completely excluded on the S-200 column, eluting entirely at the same volume as unlabeled AuNPs, despite the presence of a similar amount of α′ (FIG. 14C). For the intermediate S-300 column, the monovalent S1-AuNPs were slightly retained, consistent with the fact that its pore size was only slightly larger than the AuNPs (FIG. 14B). Since all other variables were held constant, these changes in retention can be attributed to the differences in exclusion characteristics of the base resins. The observation that S1-AuNP retention decreased dramatically as the pore size of the base support approached the AuNP diameter strongly supports the theory that size-exclusion, in addition to the affinity interaction, modulates retention in DNA valency sorting.

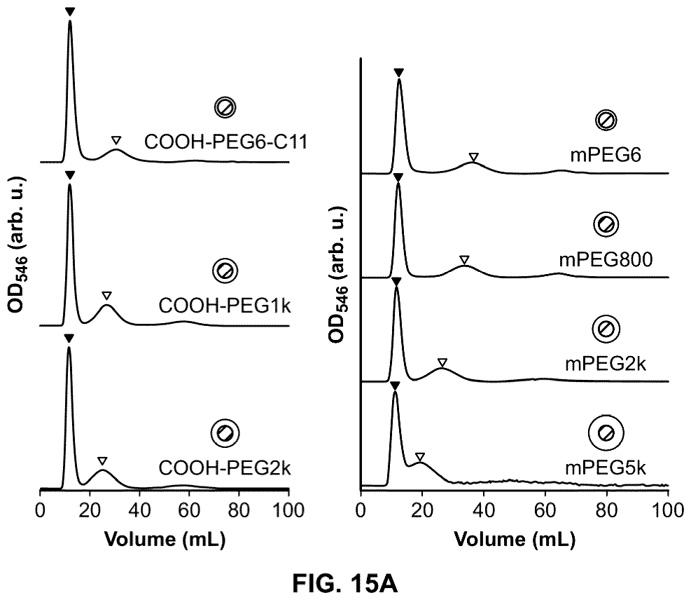

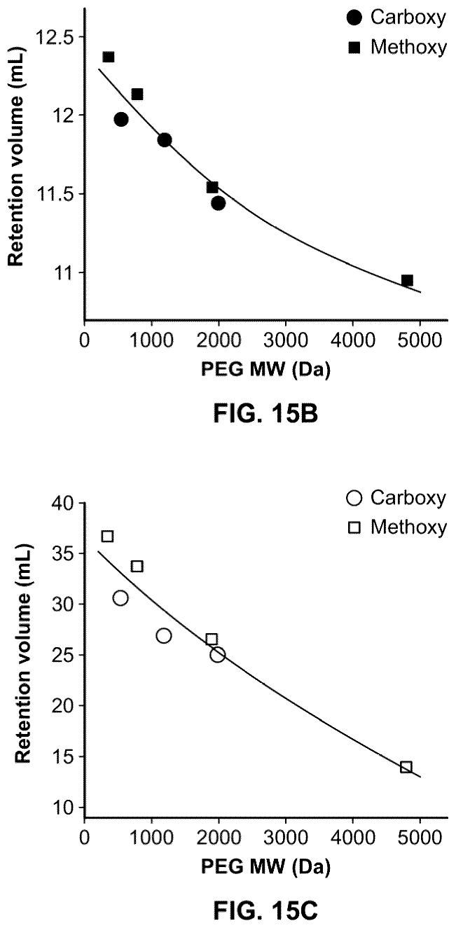

Effect of ligand coating. Next, it was investigated how the identity of the ligand coating affected the outcome of DNA valency sorting. AuNP solutes were prepared with PEG ligands comprising a range of MW and two chemically distinct terminal functional groups. Representative chromatograms of 20 nm S1-AuNPs coated with carboxy-terminated PEGs 2, 4and 5 (Table 2) are shown in FIG. 15A. Also seen in FIG. 15 are a set of chromatograms acquired of similar AuNPs coated with methoxy-terminated PEGs 1, 3, 6 and 7.

The retention volumes of the unlabeled and monovalent peaks (filled and open arrows, respectively) are plotted as a function of PEG MW in FIGS. 15B and 15C. Importantly, it is found that samples coated with methoxy-and carboxy-terminated PEGs behaved similarly on the column. The similarity between these two data sets led to the conclusion that the terminal functional group had little detectable effect on retention, at least among neutral- or negatively-charged groups. This result is consistent with the expected specificity of the affinity interaction, which should render DNA valency sorting immune to variations in nanoparticle surface chemistry.

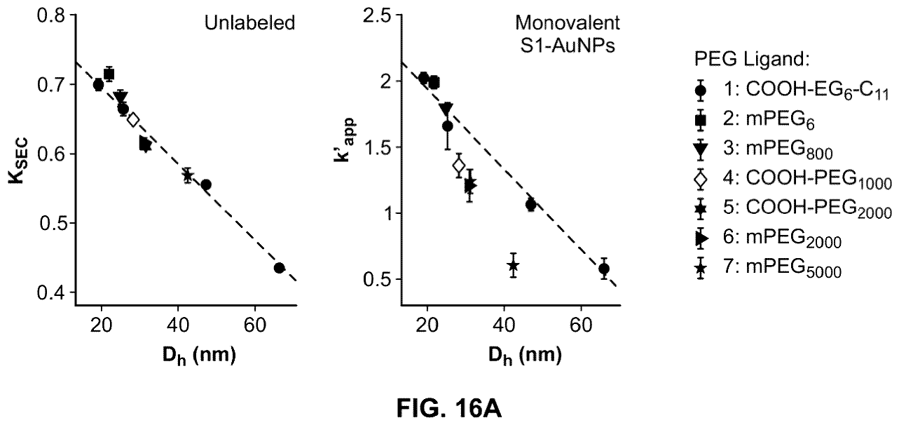

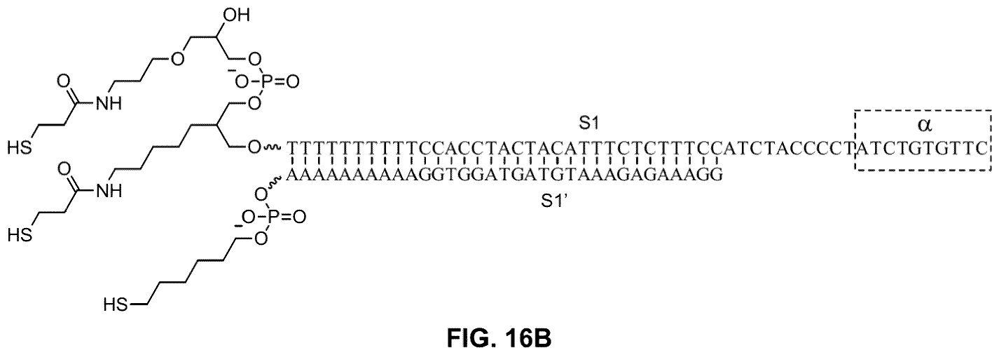

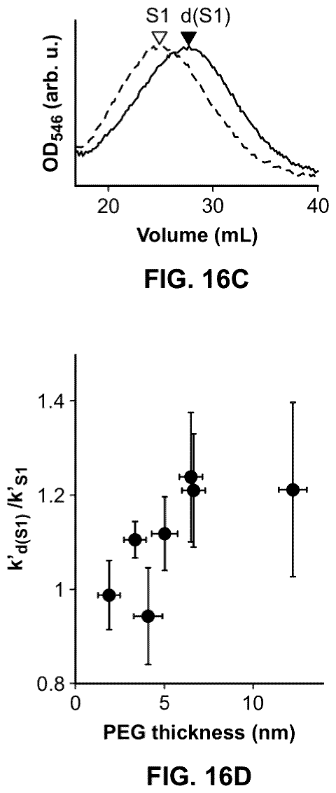

Another important observation in FIGS. 15B and 15C is that retention decreases with increasing PEG MW. DLS measurements revealed that Dₕ increased from 21.6 nm to 40.9 nm as PEG MW increased from 356 to 4800 Da (Table 3). Following our understanding of the relationship between retention and diameter developed earlier, AuNPs should experience greater exclusion effects as PEG MW increases due to their larger size, leading to a decrease in retention volume for both unlabeled and monovalent species. To evaluate whether the AuNPs coated with different PEG exhibited the expected exclusion effects, the size exclusion chromatography distribution coefficients, K_{SEC}, of the unlabeled peaks were plotted against hydrodynamic diameter, Dₕ (FIG. 16A). Data from unlabeled 10-60 nm AuNPs coated with PEG 2 were included as well for comparison. As shown, all data points followed the same linear trend with Dₕ, independent of the PEG ligand. Consequently, PEG ligands alter the exclusion behavior of AuNPs in a manner consistent with the size-exclusion characteristics of the resin.

It was next investigated whether the exclusion effect also accounted for the observed decrease in monovalent S1-AuNP retention with increasing PEG MW (FIG. 15C). The apparent retention factor was plotted versus Dₕ and was calculated as k′ₐₚₚ=(Vᵣ−Vₙᵣ)/Vₙᵣ, where Vᵣ is the retention of the interacting species and Vₙᵣ is the retention of a solute with equivalent exclusion characteristics which does not interact with the stationary phase. Conveniently, in this system, Vₙᵣ is given by the retention of the equivalent unlabeled AuNPs which can be obtained in the same chromatogram. Data for 20 nm S1-AuNPs coated with various PEG ligands was compared to similar data for 10-60 nm S1-AuNPs coated with a single PEG ligand, PEG 2 (FIG. 16A). As both data sets contained samples with the same range of diameters, it was expected that if the retention is completely determined by size-exclusion effects, then all data points in the k′ₐₚₚ versus Dₕ plot should fall on a common curve.

As shown in FIG. 16A, the two data sets did not follow a common trend. K′ₐₚₚ for S1-AuNPs coated with PEG 2 varied linearly with Dₕ over the entire range of diameters investigated. A linear fit to this data set is plotted as the dashed line (it is noted that the linear fit is empirical, not based on any underlying physical model). For S1-AuNPs coated with PEG of MW≤800 Da, K′ₐₚₚ values closely followed the empirical trend line; however, for higher MW PEG, the data points appeared to follow a different trend, and k′ₐₚₚ values were significantly lower than what would be expected for a monovalent S1-AuNP of that diameter coated with PEG 2. These results indicated that the exclusion effect alone was not sufficient to describe the retention behavior of monovalent S1-AuNPs coated with any arbitary PEG ligand. Therefore, PEG must exert an additional, as-yet unidentified effect on retention.

One possibility is that PEG could affect S1-AuNP retention by altering the affinity interaction between α and α′. Comparison between the thickness of the PEG coating and the length of 53-nt strand S1 suggests that steric occlusion of the affinity interaction is a possibility in DNA valency sorting. For example, as PEG MW increased from 356 to 4800 Da, the apparent thickness of the coating on the AuNP increased from 1.9 to 12.2 nm (Table 4); some of these values are comparable or larger than the estimated root-mean-square (rms) end-to-end distance (Rivetti, Walker, and Bustamante 1998) of strand S1 (˜9 nm). Therefore, it is possible that the PEG coating partially or fully occludes the keyword α at the end of S1. Such a steric barrier could prohibit productive interaction with the stationary phase, weakening the affinity interaction and leading to a decrease in the retention volume.

TABLE 4
Estimated thickness of indicated
PEG ligands on 20 nm AuNPs.

| Identity | MW (Da) | Thickness (nm)^{a} |
|---|---|---|
| 1 | 356 | 1.9 ± 0.6 |
| 2 | 520 | 4.1 ± 0.8 |
| 3 | 791 | 3.3 ± 0.6 |
| 4 | 1200 | 5.0 ± 0.7 |
| 5 | 2000 | 6.7 ± 0.7 |
| 6 | 1900 | 6.5 ± 0.7 |
| 7 | 4800 | 12.2 ± 0.8 |

^{a}Determined by subtracting TEM diameter from DLS hydrodynamic diameter and dividing by 2.

The steric barrier theory predicts that the effect of PEG on the retention volume would be mitigated if the DNA strand containing α were longer, extending sufficiently beyond the PEG layer. To test this theory, strand S1 was extended by introducing a 33-bp double-stranded (ds) DNA region at the 3′-end, where it is anchored onto the AuNP. The dsDNA region was formed by hybridizing a complementary sequence S1′ to S1 to form the 2-strand complex d(S1) (see FIG. 16B). 33-nt S1′ was also modified with a single alkylthiol group at its 5′-end to anchor it onto the AuNP surface and minimize the possibility of denaturation during chromatography. 20-nt at the 5′-end of S1, including the 10-nt keyword α, were left as an ssDNA overhang. The 10-nt ssDNA spacer before the keyword should prevent any modulation of the affinity interaction due to coaxial stacking interactions at a nick site. Including the dsDNA region was estimated to increase the rms end-to-end distance of the attached DNA strand to ˜14 nm, longer than the PEG ligands tested here.

The chromatography study was repeated with 20 nm d(S1)-AuNPs coated with PEGs 1-7 to examine how retention of the monovalent peak changed when the length of the attached DNA strand was increased. Chromatograms for S1- and d(S1)-AuNPs coated with PEG 5 (zoom of the monovalent peak) are shown in FIG. 16C as a representative example of the results. In general, as in this example, the retention volume of d(S1)-AuNPs increased relative to S1-AuNPs. This observation suggested that introducing a dsDNA region in d(S1) strengthened the affinity of the monovalent species, consistent with the predicted effect of extending a beyond the PEG layer. More quantitatively, d(S1)-AuNPs exhibited k′ₐₚₚ values which closely followed the trend established by S1-AuNPs coated with PEG 2. The one exception to this trend was d(S1)-AuNPs coated with PEG 7, possibly due to its thickness (12.2 nm), which approached the estimated length of d(S1) (˜14 nm). In general, however, these results suggested that, in contrast to S1-AuNPs, d(S1)-AuNP retention was dominated primarily by size-dependent exclusion effects and, therefore, that inclusion of the dsDNA region largely reversed the effect of PEG ligands on the affinity interaction.

Ultimately, comparison to the S1-AuNPs coated with PEG 2 is inherently arbitrary, as the retention of these samples may also be influenced by the PEG ligand. To characterize the effect of PEG thickness on retention without comparison to an arbitrary data set, the ratio of k′ₐₚₚ for d(S1)-and S1-AuNPs (k′_{d(S1)}/k′_{S1}) was evaluated for each PEG ligand individually. Since the two solutes have similar exclusion characteristics, this quantity more closely reflects how the strength of the affinity interaction was modulated by extending the length of S1. FIG. 16D indicates that the ratio k′_{d(S1)}/k′_{S}1 is positively correlated with PEG thickness, increasing from ˜1.0 for the shortest PEG to a maximum of 1.25 for PEGs 5-7. This trend suggests that extending the DNA strand had a minimal effect for small PEG ligands, and a much larger effect for thicker PEG coatings. As such, it provides strong evidence in support of the steric barrier theory, which predicts that thicker PEG layers present a greater steric barrier and therefore exert a greater influence on an affinity interaction.

Application of experimental insights to improve resolution. It is clear that both diameter and surface coating of the nanoparticle exert a significant deleterious effect on separation outcome. A practical consequence of these effects is that they limit the types of nanoparticle-bioconjugates which can be effectively purified. For example, using an initial chromatography method, resolution dropped precipitously for large diameter S1-AuNPs (see, e.g., FIG. 13A), suggesting—at least initially—that DNA valency sorting may not be applicable for nanoparticles larger than ˜20 nm. Yet, the understanding that size and surface coating influence separation outcome through size-exclusion and steric occlusion, respectively, should inform practical strategies to improve the chromatography result. For example, in the general case, it may be beneficial to maximize the pore size of the base resin and minimize the thickness of the stabilizing ligand coating. For the specific case of DNA valency sorting, which relies on the highly programmable association of two DNA oligonucleotides, a more straightforward approach should be possible: since both size and surface coating ultimately perturb the α-α′ interaction, the strength of which can be modulated by mobile phase concentrations (e.g., [NaCl]), we reasoned that their deleterious effects could potentially be counteracted by careful choice of mobile phase composition.

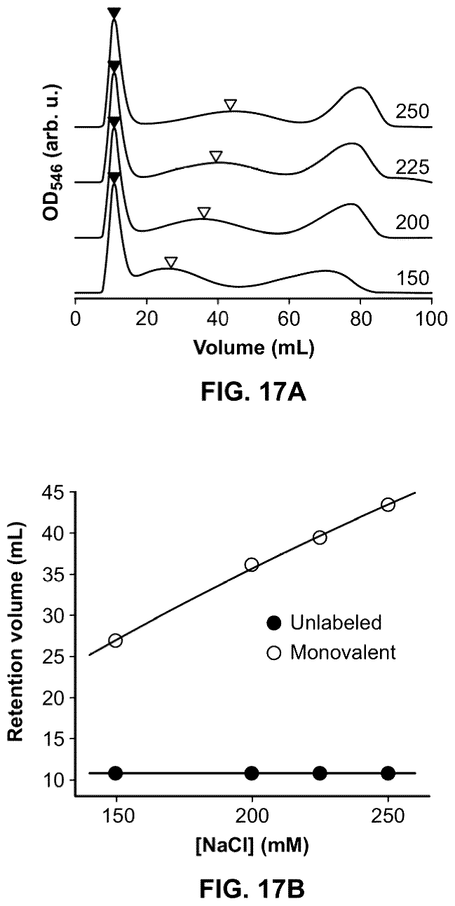

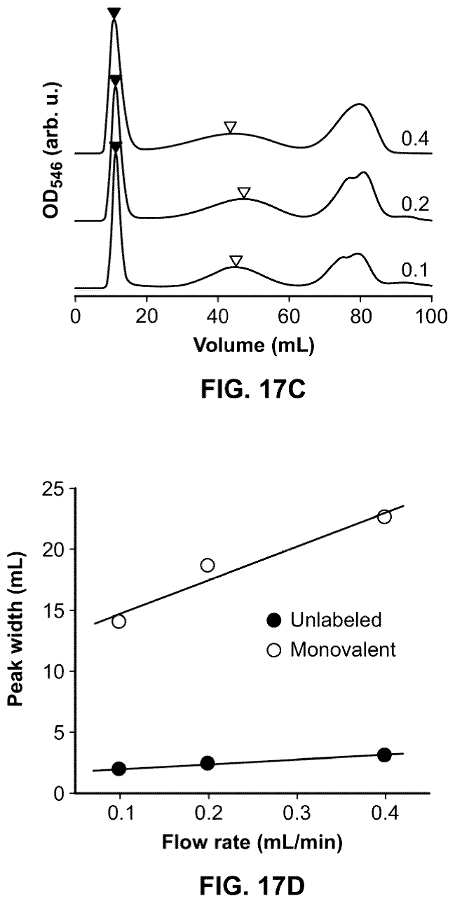

The chromatography method was changed in order to improve resolution of larger diameter S1-AuNPs. First, the effect of increasing initial [NaCl] in the mobile phase was investigated. Higher [NaCl] will increase the affinity constant of the α-α′ interaction, thereby increasing the retention of all S1-AuNPs and counteracting the negative consequences of larger AuNP sizes. 60 nm S1-AuNPs stabilized with PEG 4 were used as a representative sample, because similar 60 nm S1-AuNPs stabilized with PEG 2 exhibited extremely low resolution separation under initial conditions. See FIG. 13A. (It is noted that the PEG ligand was changed to maintain colloidal stability in higher [NaCl].) Chromatograms collected at initial [NaCl] of 150, 200, 225, and 250 mM are shown in FIG. 17A. As anticipated, the higher initial [NaCl] increased the retention volume of the monovalent S1-AuNPs, but did not affect that of the unlabeled species (FIG. 17B), resulting in a slight improvement of resolution between the two peaks.

It was also apparent in the chromatograms that increased [NaCl] also increased peak width, suggesting that adjusting mobile phase composition alone would be insufficient to maximize resolution. To address peak dispersion, the flow rate was also investigated. Chromatograms of 60 nm S1-AuNPs coated with PEG 4 obtained at an initial [NaCl] of 250 mM and flow rates of 0.4, 0.2, and 0.1 ml/min are shown in FIG. 17C. Reducing the flow rate produced an approximately linear decrease in peak dispersion for both unlabeled and monovalent species, and no minimum was observed in the plot shown in FIG. 17D. The combined effects of increasing initial mobile phase [NaCl] and decreasing the flow rate substantially increased the average resolution of monovalent 60 nm S1-AuNPs. At [NaCl]≥200 mM and a flow rate of 0.1 ml/min, the average resolution of the monovalent peak exceeded baseline resolution (1.5). The same procedure was also used to achieve baseline or near-baseline resolution of monovalent 40 and 80 nm diameter S1-AuNPs.

These examples demonstrate that with only initial [NaCl] in the mobile phase and flow rate, the chromatography result could be sufficiently improved to permit preparative purification of monovalent S1-AuNPs with diameters as large as 80 nm. Certainly, the specific manifestation of this approach is unique to DNA valency sorting. Yet, the strategy was informed by the underlying mechanisms by which size and surface coating impact resolution. Since these mechanisms are general to any liquid chromatography procedure, we anticipate that they will be similarly useful for guiding the improvement of other chromatography techniques.

### Example 6

Achieving a more favorable balance between analysis time and scope is crucial to routinize the purification process, enable rapid analytical characterization, and permit more widespread application of these specialized reagents. A variety of modes of liquid chromatography have been explored for purifying discrete DNA-nanoparticle conjugates, but comparatively less attention has been paid to the characteristics of the media used in separation. All techniques to date have relied on adapting existing media from biomolecule purification, which has disadvantages when applied to nanomaterials. Primarily, these conventional beaded resins are designed for protein separation and have relatively small pore sizes compared to the size range of nanoparticle solutes. This has negative consequences for resolving discrete DNA-nanoparticle conjugates; for example, in DNA valency sorting chromatography, it has been found that resolution and binding capacity deteriorated when nanoparticles were excluded from the pore volume, a phenomenon which is also observed for biological macromolecules in conventional affinity or anion-exchange chromatography.

One appealing solution is to use monolithic rather than conventional packed bed supports. Monolithic columns consist of a continuous polymeric or silica phase in a column tube that which contains large so-called “through-pores” with diameters of ˜1 μm to allow mobile phase to pass through the column. The large pore sizes improve capacity for large solutes like viruses and plasmids, and the lack of a traditional void volume facilitates rapid, convective transport to the stationary phase, improving mass transfer rates and, in many cases, resulting in flow-rate-independent resolution. Another advantage of monolith columns is that they can be produced in extremely small sizes, equivalent to a typical membrane disk, presenting the possibility for rapid analysis with low buffer consumption.

Here, it is investigated whether the large throughpores and reduced size of a monolith bed could be used to improve the efficiency and speed of DNA valency sorting chromatography. It is found find that, compared to a conventional packed bed column, the monolith format provides a modest reduction in analysis time for separations of small nanoparticles, but a massive improvement in time required for separation for larger nanoparticles. Further, while the large pore size of the monolith bed does not completely eliminate solute size-dependent effects, it reduces them to the point that a single common method, requiring a little over an hour in analysis time, can be applied to resolve discrete DNA-nanoparticle conjugates in a wide range of sizes. It is also demonstrated how the advantages of the monolith format extends DNA valency sorting to new applications, including rapid analytical screening of DNA-nanoparticle conjugates, the purification of fragile nanoparticles like liposomes, and the solid phase, flow-through synthesis of nanostructures.

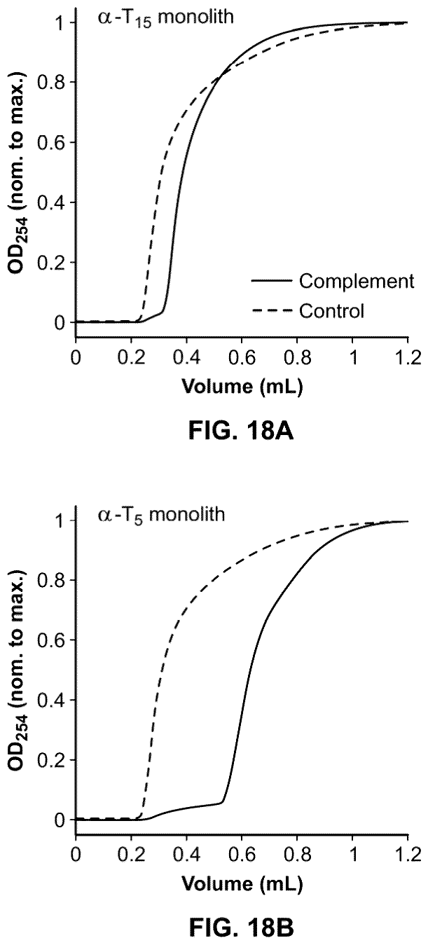

Preparation of monolith DNA valency sorting columns. A monolith DNA valency sorting column was prepared by coupling 3′-amine terminated capture sequence α′-T₁₅ onto a commercially available 0.1 mL carbonyldiimidazole-activated methacrylate monolith disk (diameter: 7 mm, length: 2.1 mm). The amount of DNA immobilized on the monolith was characterized by frontal analysis of the breakthrough of a complementary DNA sequence α (sequence: 5′-CTTGTGTCTA-3′) [SEQ ID NO. 1]. Comparison to the breakthrough of a non-complementary DNA sequence, T₂₀, positively indicated α binding and therefore the presence of the capture sequence on the monolith (FIG. 18A).

Quantitatively, the dynamic binding capacity at 10% breakthrough was determined to be 1.51 nmol, corresponding to an approximate concentration of 15.1 nmol mL⁻¹. This value is similar to that attained on the prior DNA valency sorting column (9-13 nmol mL⁻¹), which was prepared from conventional low pressure size-exclusion chromatography media.

A second monolith disk was prepared in addition to examine the effect of the concentration of the capture sequence on performance. To increase the immobilization density on the monolith, the poly-T spacer was decreased to five nucleotides, and a higher concentration of sodium sulfate was included during the coupling reaction. As indicated by the breakthrough curve in FIG. 18B, these changes did substantially increase the amount of α′ immobilized on the monolith. The dynamic binding capacity at 10% breakthrough for the α′-T₅-modified monolith was 5.53 nmol, corresponding to a concentration of 55.3 nmol or 0.26 mgmL⁻¹, similar to the maximum density of DNA immobilization on monoliths quoted in the literature.

To perform these tests, 0.1 mL monolith disks modified with α′-T₁₅ (18A) or α′-T₅ (18B) DNA were exposed to 20 M complementary DNA sequence a (5′-CTTGTGTCTA-3′; Complement) or 4 M non-interacting DNA composed of 20 T nucleotides (T₂₀; Control) at flow rate of 0.5 mL min⁻¹ in 1× TE buffer containing 1 M NaCl. The delay in breakthrough of sequence a in both monoliths compared to T₂₀ confirmed the presence of immobilized α′.

Preliminary application of DNA valency sorting to the monoliths. After successful conjugation of the capture sequence, DNA valency sorting was performed with the monoliths. 5 nm diameter gold nanoparticles (AuNPs) sparsely labeled with bis(alkyl thiol)-modified 53-nucelotide DNA strand S1 and stabilized with a thiol-modified methoxy-polyethylene glycol (mPEG) ligand were used as a representative sample. Strand S1 contains the complementary keyword sequence α at the 5′-end for interaction with the capture sequence immobilized on the columns.

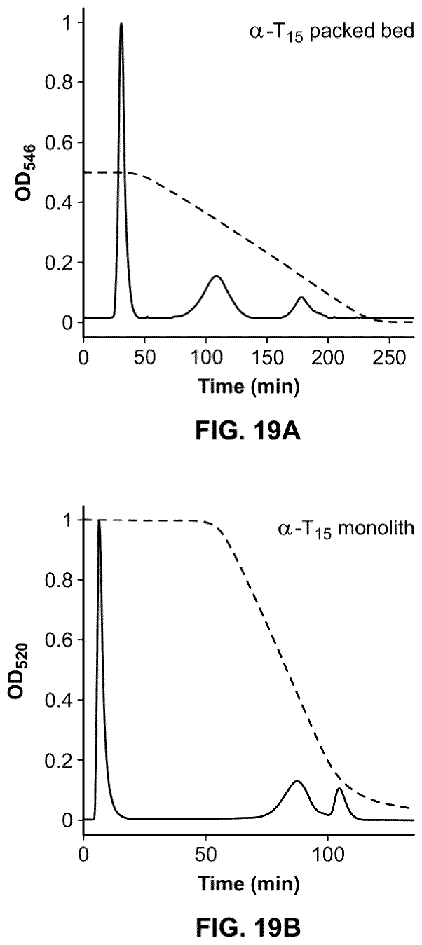

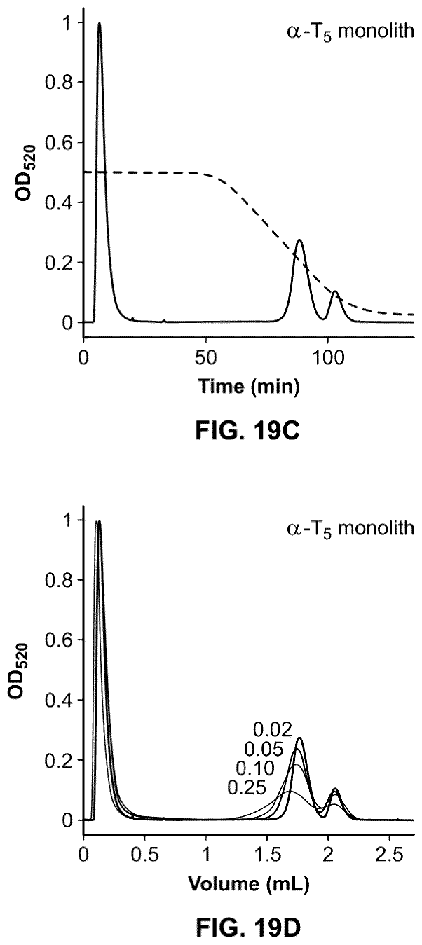

The result of analyzing this sample on the conventional DNA valency sorting column using a standard method is shown in FIG. 19A. A standard method was used; after sample injection (0.1 mL), the start buffer (TE, 0.01% SDS, 100 mM NaCl) was changed to elution buffer (TE, 0.01% SDS) over a 75 mL linear gradient at a flow rate of 0.4 mL/min.

Here, the input sample contained three resolved peaks, which can be assigned to AuNPs bearing zero, one, and two or more strands of S1.

FIGS. 19B and 19C show results obtained for DNA valency sorting separations of 5 nm S1-AuNPs carried out on the α′-T₁₅ and α′-T₅ monoliths, respectively.

For FIG. 19B, after sample injection (0.01 mL), the start buffer (TE, 0.01% SDS, 0.1% Tween-20, 200 mM NaCl) was changed to elution buffer (TE, 0.01% SDS, 0.1% Tween-20) over a 1 mL linear gradient at a flow rate of 0.02 mL/min. For FIG. 19C, after sample injection (0.01 mL), the start buffer (TE, 0.01% SDS, 0.1% Tween-20, 100 mM NaCl) was changed to elution buffer (TE, 0.01% SDS, 0.1% Tween-20) over a 1 mL linear gradient at a flow rate of 0.02 mL/min. For FIGS. 19B and 19C, the linear mobile phase gradient was visualized by repeating a blank chromatography run with start buffer containing a 0.1% acetone and monitoring the elutant absorbance at 260 nm.

Since the gradient conditions from the conventional column cannot be directly translated to the monoliths, suitable conditions were used based on a brief empirical screening. This initial data set indicates that both monolith columns are able to attain separation between the distinct DNA valencies present in the input sample, suggesting that they can effectively perform DNA valency sorting; however, the resolution observed under these conditions is not yet equivalent to that attained on the original conventional column. Nonetheless, even under these preliminary conditions it is possible to observe some of the benefits of the monolith format. For example, the time required for analysis (˜125 min vs. ˜250 min) was reduced by 50% compared to the conventional packed bed column.

Preliminarily, we also found that not all potential benefits of the monolith format appeared to translate to DNA valency sorting. For example, one documented advantage in ion exchange or hydrophillic interaction chromatography on monoliths is their flow-rate-independent resolution. This arises from the rapid convective mass transfer to the stationary phase which is achieved with the large throughpores in the monolith bed. FIG. 19D shows separations of the same sample on the α′-T₅ monolith under identical gradient conditions as FIG. 19C, but at different flow rates. These data indicate that the peak width, and therefore the resolution, in DNA valency sorting on the monolith is not independent of flow rate. This result can be rationalized by considering contributions to peak broadening other than mass transfer. Due to their specificity, affinity interactions generally have much lower association rate constants compared with ionic or hydrophobic interactions. In the monolith format, it is likely that this factor begins to dominate peak broadening.

Resolution of monoliths compared to conventional column. The preceding experiment indicated qualitative benefits of the monolith over the conventional DNA valency sorting column. For a quantitative comparison of performance, the height equivalent of a theoretical plate (HETP) for linear gradient elution, HETP_{LGE}, was evaluated for each column as a function of linear mobile phase velocity, u. HETP_{LGE} can be obtained experimentally by analyzing a single sample on a given column while varying values of u and g, the slope of the gradient in M mL⁻¹. As before, a representative sample of mPEG-stabilized 5 nm S1-AuNPs was used for the analysis, looking specifically at the monovalent peak because it is the best resolved species in the separations.

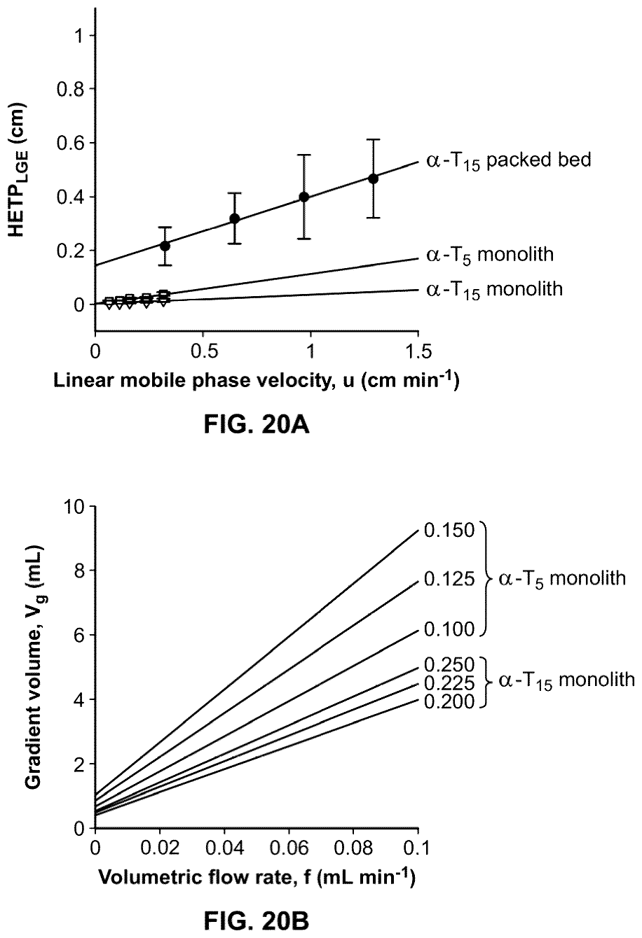

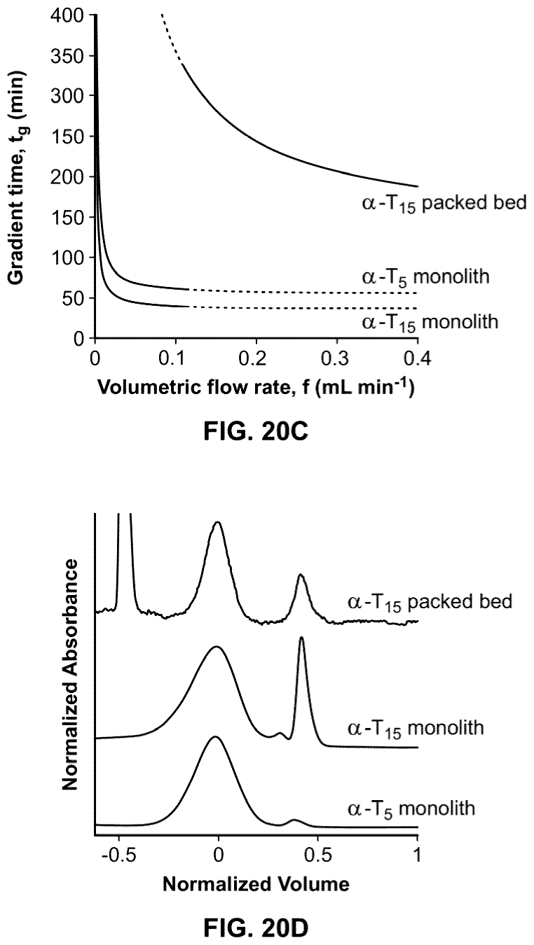

HETP_{LGE}-u curves obtained for the mPEG-stabilized monovalent 5 nm S1-AuNPs on the three columns are summarized in FIG. 20A. The analysis reveals that the HETP_{LGE} for the conventional packed bed column is substantially larger than for either monolith column at all linear mobile phase velocities investigated. It is noted that the range of mobile phase velocities differed between the conventional column and the monoliths, because u>0.4 cm/min on the monolith produced inconsistent results. This effect was likely due to the monolith bed height, which was too short to reach steady-state conditions at high flow rates. Direct comparison can be made at one common mobile phase velocity (u≈0.3 cm/min). Under this condition, HETP_{LGE} was 0.2 cm on the packed bed column, 0.01 cm on the α′-T₁₅ monolith, and 0.04 cm on the α′-T₅ monolith. Overall, this suggests that the monolith format improves dramatically on the efficiency of DNA valency sorting.

It was also observed that the monoliths exhibited a shallower slope to the HETP_{LGE}-u curve than the conventional packed bed column. This result is consistent with the expectation that convective flow in the monolith will minimize or eliminate contributions from mass transfer to peak broadening. Therefore, while the outcome of DNA valency sorting on monoliths depends on flow rate (as qualitatively observed in FIG. 19D), this data reveals that the dependence is weaker than on the conventional column.

Using the experimental HETP_{LGE}-u curves, it was investigated what gradient and flow rate conditions would be required to attain equivalent resolution on the monoliths as on the packed bed column. Under linear gradient elution, equivalent resolution is obtained on different columns when the dimensionless factor O, given by

$$O = \frac{{ZI}_{a}}{g \times {V}_{0} \times {HETP}_{LGE}} , \qquad ( Eq . 3 )$$

is equivalent, where Z is the column height in cm, Iₐ is a normalization factor with a value of 1, g is the gradient slope in M/mL, and V₀ is the void volume of the column in mL. O was calculated for the standard conditions found to achieve adequate separation of the mPEG-stabilized 5 nm S1-AuNPs on the conventional column (e.g. FIG. 19A). FIG. 20B shows plots of V_{g}, the gradient length, required to achieve equivalent resolution on the two monoliths for the volumetric flow rates f and initial NaCl concentrations indicated. The two monoliths achieve equivalent resolution at distinct conditions, despite their identical geometrical characteristics, due to the small differences in efficiency indicated in the HETP_{LGE}-U curves of FIG. 20A. The improved efficiency of either monolith permits equivalent separations to be performed with much steeper gradients and much lower buffer consumption than on the conventional column.

The monolith format should also significantly speed up the DNA valency sorting operation compared to the conventional column. FIG. 20C plots t_{g}, the time of the gradient portion of the analysis, required to achieve equivalent resolution as a function of volumetric flow rate f for the two monoliths. For comparison, t_{g} for the iso-resolution separation on the conventional column is also shown. It is clear from the comparison of the three curves that at all experimentally relevant flow rates, separation on the monoliths proceeds remarkably faster. For example, at the common volumetric flow rate of 0.1 mL/min, t_{g} is 4-7-fold lower on the monoliths compared to the conventional column.

These predictions were tested experimentally by performing equivalent resolution separations of 5 nm S1-AuNPs on the two monolith columns using parameters determined from FIG. 20B. A comparison of these separations to the reference chromatogram obtained on the conventional packed bed column is shown in FIG. 20D. To facilitate comparison despite the varying extra-column volumes in these systems, the elution volume was adjusted such that the retention volume of the monovalent peak was zero, followed by normalization to the total volume of the chromatography program. As shown in the plot, the resolution between the eluted peaks on the monolith appears qualitatively similar to that on the packed bed column, confirming that the original DNA valency sorting method could be adequately transferred to the monoliths using the iso-resolution formalism of Eq. 3.

Size dependence on the monolith. Next it was tested if the large throughpores in the monolith would overcome the size-dependent exclusion effects observed on the previous conventional packed bed column.

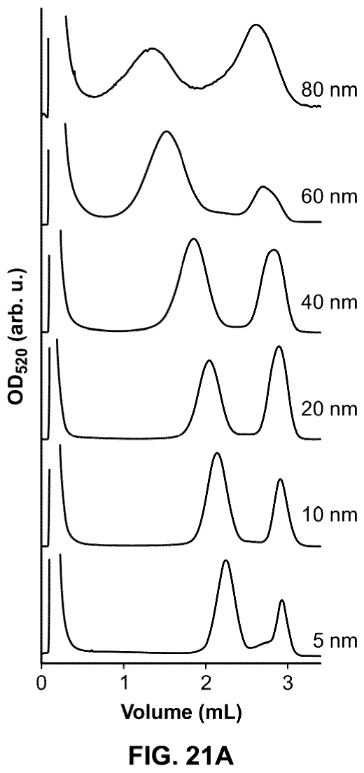

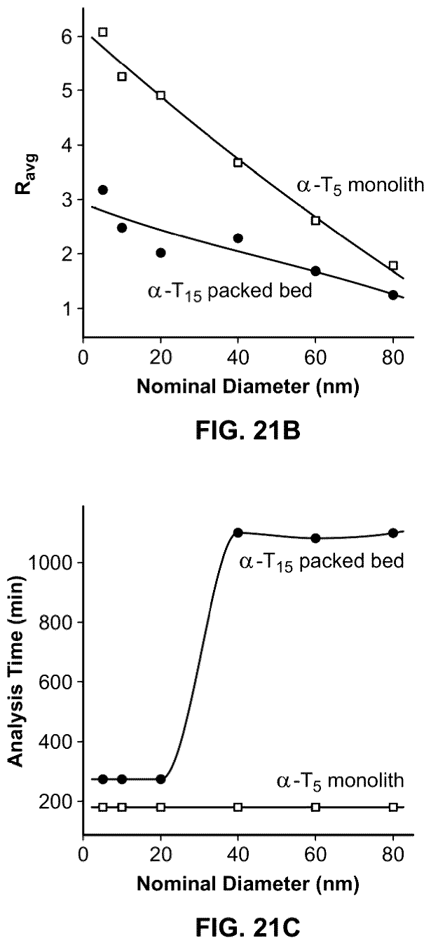

FIG. 21A shows chromatograms of S1-AuNPs stabilized with a carboxy-termined PEG ligand (MW 1000; COOH-PEG₁₀₀₀) obtained on the α-T₅ monolith. The chromatography method ([NaCl]=125 mM, g=0.0625 M/mL, V_{g} =2 mL, f=0.02 mL/min) was designed using Eq. 3 to have equivalent resolution as the conventional column separation of mPEG-coated 5 nm S1-AuNPs (e.g., FIG. 19A). As shown in the plots, there are obvious changes in both retention and peak width as nanoparticle diameter increases, similar to observations on the conventional packed bed column. This result suggests that solute size still impacts the separation outcome even on the monolith column. Because exclusion effects are substantially mitigated on the monolith, it is most likely that this phenomena is kinetic in origin, although further experimentation is needed to ascertain the precise cause of these effects.

Notably, well-resolved peaks were observed for all S1-AuNP sizes with a single chromatography method on the α-T₅ monolith (see FIG. 21A). FIG. 21B plots the average resolution of the monovalent peak, R_{avg}, as a figure of merit for the quality of the separation. For all diameters R_{avg} exceeds ˜1.5, which is typically defined as baseline resolution. This was distinct from similar analyses performed on the conventional column; when using a single equivalent gradient method, resolution deteriorated rapidly as nanoparticle size exceeded ˜20 nm. To overcome this effect, the process previously included optimized separate chromatography methods to attain baseline or near-baseline resolution of larger nanoparticles on the conventional column. R_{avg} for these optimized methods is also plotted as a function of nanoparticle sizes in FIG. 21B, indicating that separations on the monolith were comparable or superior to these previously optimized methods on the conventional column for all diameters investigated.

Further, as seen in FIG. 21C, the analysis time required for a monolithic bed remains substantially constant as the nominal diameter increases, which differs greatly from the conventional packed beds, where analysis time increases greatly at larger diameters.

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Those skilled in the art will recognize or be able to ascertain using no more than routine experimentation many equivalents to the specific embodiments of the invention described herein. Such equivalents are intended to be encompassed by the following claims.

What is claimed:

1. A method for sorting nanoparticles or molecules, comprising:
providing a plurality of nanoparticles or molecules, each nanoparticle or molecule comprising a keyword sequence, each keyword sequence appended onto a DNA sequence attached to the nanoparticle or molecule;
allowing the keyword sequences on each nanoparticle or molecule to bind to a capture sequence coupled to a solid support substrate, each capture sequence being a reverse complement of the keyword sequence; and
releasing the nanoparticle or molecule on the solid support substrate based on a mobile phase strength of a mobile phase passing over the solid support substrate.

2. The method according to claim 1, wherein at least one of the plurality of nanoparticles or molecules comprises a plurality of keyword sequences coupled to the nanoparticle or molecule.

3. The method according to claim 2, wherein the plurality of keyword sequences are the same keyword sequence.

4. The method according to claim 2, wherein at least one of the plurality of keyword sequences is different from another of the plurality of keyword sequences.

5. The method according to claim 1, wherein at least one of the plurality of nanoparticles or molecules comprises a plurality of different molecules, each molecule coupled to a different keyword sequence.

6. The method according to claim 1, further comprising determining at least one valency of the plurality of nanoparticles or molecules based on a retention time, volume of mobile phase utilized, or a combination thereof.

7. The method according to claim 1, further comprising determining an identify of the molecule based on based on a retention time, volume of mobile phase utilized, or a combination thereof.

8. The method according to claim 1, further comprising determining an identify of the molecule based on based on a retention time, volume of mobile phase utilized, or a combination thereof.

9. The method according to claim 1, wherein the mobile phase strength is modulated in a linear manner.

10. The method according to claim 9, wherein modulating the mobile phase strength comprises decreasing the mobile phase concentration of NaCl in a linear gradient.

11. The method according to claim 1, wherein the mobile phase strength is modulated in a non-linear manner.

12. The method according to claim 1, wherein the keyword sequence is a 5-nt to 20-nt sequence.

13. The method according to claim 1, wherein the solid support substrate is an exclusion chromatography resin.

14. The method according to claim 13, wherein the capture sequence is grafted to the exclusion chromatography resin via carbonyldiimidazole coupling chemistry.

15. The method according to claim 1, wherein the solid support substrate is a monolithic support.

16. The method according to claim 1, wherein the nanoparticle or molecule is a gold nanoparticle, a silver nanoparticle, an iron oxide nanoparticle, a semiconducting nanocrystal, a gold nanorod, a small molecule, a ligand, a protein, or an antibody.

17. The method according to claim 1, further comprising injecting a first buffer into a column, then injecting a sample containing the plurality of nanoparticles into the column.

18. The method according to claim 1, further comprising collecting fractions, pooling the fractions, and concentrating the pooled fractions.

19. The method according to claim 1, further comprising determining a UV-Vis spectra of a sample containing a released bound nanoparticle.

20. The method according to claim 1, further comprising determining an optical density of a sample containing a released bound nanoparticle.

21. The method according to claim 1, further comprising determining a fluorescence of a sample containing a released bound nanoparticle.

22. The method according to claim 1, further comprising determining a refractive index of a sample containing a released bound nanoparticle.

23. The method according to claim 1, wherein the nanoparticles are at least partially coated with a coating material.

24. The method according to claim 1, wherein the coating material is a polyethylene glycol (PEG).

25. The method according to claim 1, further comprising washing the solid structure with a solvent after releasing the bound nanoparticles.

26. The method according to claim 1, wherein at least one additional material is bound to the nanoparticle.

27. The method according to claim 26, wherein the at least one additional material is an additional nanoparticle or a biomolecule.

28. The method according to claim 1, further comprising:
collecting the plurality of nanoparticles or molecules after release; and
drying and/or purifying the collected plurality of nanoparticles or molecules, where each nanoparticle or molecule is a colored, magnetic, or photoluminescent nanoparticle, a small molecule, or a biomolecule, and each nanoparticle or molecule bears a single bio-active molecule or reactive group.

29. The method according to claim 1, wherein each nanoparticle or molecule comprising a plurality of different keyword sequences coupled to the nanoparticle or molecule.

30. The method according to claim 29, wherein each different keyword sequence is coupled to one or more DNA sequences.

31. The method according to claim 30, wherein each different keyword sequence is coupled to a plurality of DNA sequences.

32. A system for DNA valency sorting chromatography, comprising:
a column packed with a solid support substrate and a solvent, the solid support substrate coupled to a plurality of capture sequences; and
a plurality of nanoparticles within the column, where at least one keyword sequence appended to a DNA sequence is attached to each nanoparticle, each keyword sequence being a complement to the capture sequence.

33. The system according to claim 32, wherein the nanoparticles are each partially coated in a coating layer.

34. The system according to claim 33, wherein the coating layer is a polyethylene glycol (PEG).

35. The system according to claim 32, wherein the solid support substrate is an exclusion chromatography resin.

36. The system according to claim 32, wherein the solid support substrate is a monolithic support.

37. The system according to claim 32, wherein the nanoparticle or molecule is a gold nanoparticle, a silver nanoparticle, an iron oxide nanoparticle, a semiconducting nanocrystal, a gold nanorod, a small molecule, a ligand, a protein, or an antibody.

38. A composition of matter, comprising:
a plurality of nanoparticles coupled together, each nanoparticle is coupled to at least one other of the plurality of nanoparticles via a nucleotide connection;
wherein each nucleotide connection independently comprises:
a keyword sequence appended to a DNA sequence attached to one of the nanoparticles being coupled; and
a capture sequence appended to a DNA sequence attached to the other of the nanoparticles being coupled, the capture sequence being a reverse complement of the keyword sequence.

39. The composition of matter according to claim 38, wherein the plurality of nanoparticles includes at least three nanoparticles.


SEQUENCE LISTING (22 sequences)

SEQ ID NO: 1 - DNA, 10 bp, synthetic construct
  source 1..10: mol_type = other DNA
cttgtgtcta

SEQ ID NO: 2 - DNA, 10 bp, synthetic construct
  source 1..10: mol_type = other DNA
tagacacaag

SEQ ID NO: 3 - DNA, 53 bp, synthetic construct
  source 1..53: mol_type = other DNA
cttgtgtctatccccatctacctttctctttacatcatccacctttttttttt

SEQ ID NO: 4 - DNA, 53 bp, synthetic construct
  source 1..53: mol_type = other DNA
ttttttttttccacctactacatcttaatcctagcttaatacttttcattctc

SEQ ID NO: 5 - DNA, 49 bp, synthetic construct
  source 1..49: mol_type = other DNA
cttgtgtctatttttttttgagaatgaaaagtattaagctaggattaag

SEQ ID NO: 6 - DNA, 40 bp, synthetic construct
  source 1..40: mol_type = other DNA
aagagaaaggtagatggggatagacacaagtttttttttt

SEQ ID NO: 7 - DNA, 51 bp, Synthetic construct
  source 1..51: mol_type = other DNA
aagagaaaggtagatggggatagacacaagtatattctaaaagttccaacc

SEQ ID NO: 8 - DNA, 51 bp, synthetic construct
  source 1..51: mol_type = other DNA
aagagaaaggtagatggggatagacacaagggttggaacttttagaatata

SEQ ID NO: 9 - DNA, 67 bp, synthetic construct
  source 1..67: mol_type = other DNA
taggttccaagagaaaggtagatggggatagacacaaggagaagaaaagtattaagctaggattaag

SEQ ID NO: 10 - DNA, 68 bp, synthetic construct
  source 1..68: mol_type = other DNA
cttaatcctagcttaatacttttcattctccttgtgtctatccccatctacctttctcttggaaccta

SEQ ID NO: 11 - DNA, 20 bp, Synthetic construct
  source 1..20: mol_type = other DNA
tttttttttttttttttttt

SEQ ID NO: 12 - DNA, 25 bp, synthetic construct
  source 1..25: mol_type = other DNA
tagacacaagttttttttttttttt

SEQ ID NO: 13 - DNA, 53 bp, synthetic construct; identical to SEQ ID NO: 3
  source 1..53: mol_type = other DNA

SEQ ID NO: 14 - DNA, 40 bp, synthetic construct; identical to SEQ ID NO: 6
  source 1..40: mol_type = other DNA

SEQ ID NO: 15 - DNA, 53 bp, synthetic construct; identical to SEQ ID NO: 4
  source 1..53: mol_type = other DNA

SEQ ID NO: 16 - DNA, 49 bp, synthetic construct; identical to SEQ ID NO: 5
  source 1..49: mol_type = other DNA

SEQ ID NO: 17 - DNA, 39 bp, synthetic construct
  source 1..39: mol_type = other DNA
cttaatcctagcttaatacttttcattctcaaaaaaaaa

SEQ ID NO: 18 - DNA, 51 bp, synthetic construct; identical to SEQ ID NO: 7
  source 1..51: mol_type = other DNA

SEQ ID NO: 19 - DNA, 51 bp, synthetic construct; identical to SEQ ID NO: 8
  source 1..51: mol_type = other DNA

SEQ ID NO: 20 - DNA, 67 bp, synthetic construct; identical to SEQ ID NO: 9
  source 1..67: mol_type = other DNA

SEQ ID NO: 21 - DNA, 68 bp, synthetic construct; identical to SEQ ID NO: 10
  source 1..68: mol_type = other DNA

SEQ ID NO: 22 - DNA, 10 bp, synthetic construct
  source 1..10: mol_type = other DNA
tttttttttt